SEQUENCE LISTING (10 sequences)

SEQ ID NO: 1 - protein, 16 aa, Artificial Sequence
  Amino acid sequence of LCDR1
RSSQSIVHSNGNTYLE

SEQ ID NO: 2 - protein, 7 aa, Artificial Sequence
  Amino acid sequence of LCDR2
KVSNRFS

SEQ ID NO: 3 - protein, 9 aa, Artificial Sequence
  Amino acid sequence of LCDR3
FQGSHVPLT

SEQ ID NO: 4 - protein, 5 aa, Artificial Sequence
  Amino acid sequence of HCDR1
DYEMH

SEQ ID NO: 5 - protein, 17 aa, Artificial Sequence
  Amino acid sequence of HCDR2
VIESETGGTAYNQKFKG

SEQ ID NO: 6 - protein, 16 aa, Artificial Sequence
  Amino acid sequence of HCDR3
EGITTVATTYYWYFDV

SEQ ID NO: 7 - protein, 112 aa, Artificial Sequence [sequence omitted]

SEQ ID NO: 8 - protein, 125 aa, Artificial Sequence [sequence omitted]

SEQ ID NO: 9 - protein, 219 aa, Artificial Sequence
  Light chain sequence
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, Artificial Sequence
  Heavy chain sequence
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2024/0254230 A1
(43) Pub. Date: Aug. 1, 2024

(54) METHOD OF TREATING UROTHELIAL CARCINOMA

(71) Applicant: SHANGHAI JUNSHI BIOSCIENCES CO., LTD., Shanghai (CN)

(72) Inventors: Sheng YAO, Shanghai (CN); Hui FENG, Shanghai (CN)

(21) Appl. No.: 18/282,856

(22) PCT Filed: Mar. 17, 2022
(86) PCT No.: PCT/CN2022/081534

§ 371 Date: Sep. 19, 2023

(30) Foreign Application Priority Data
Mar. 19, 2021 (WO) PCT/CN2021/081676

(51) Int. Cl. C07K 16/28; A61P 35/00

(52) U.S. Cl. CPC C07K 16/2818; A61P 35/00; C07K 2317/24

(57) ABSTRACT

The present invention discloses a method of treating a patient suffering from locally advanced or metastatic urothelial carcinoma, comprising determining a tumor mutational burden of the patient; identifying a candidate exhibiting a high tumor mutational burden, wherein the high tumor mutational burden is ≥10 mutations/Mbp; and administering to the candidate a therapeutically effective amount of toripalimab. The present invention also discloses a method of identifying a candidate having mutations in one or more of the following genes occurred in tumor cells: SMARCA4 and RB1.

### TECHNICAL FIELD

The present invention is in the field of treatment of cancer. More specifically, the present invention relates to a method of treating a patient suffering from urothelial carcinoma. The present invention also relates to a method of predicting the response of a patient suffering from urothelial carcinoma to the treatment.

### REFERENCE TO SEQUENCE LISTING SUBMITTED ELECTRONICALLY

The sequence listing of the present application is submitted electronically via Patent Center as an ST.25 TXT formatted sequence listing with the file name “0135393-0777126_SequenceListing.txt”, with a creation date of Apr. 20, 2024, and a file size of 9,472 bytes. The sequence listing is incorporated herein by reference in its entirety.

### BACKGROUND OF THE INVENTION

Patients with locally advanced or metastatic urothelial carcinoma (mUC) have poor prognosis with 5-year survival rate of only around 15%. Platinum-based chemotherapy remains the first-line standard of care for mUC. While approximately 50% of patients will have an initial response to platinum-based chemotherapy, the duration of response is short-lived. Second-line chemotherapy has only limited effect with response rate around 10% as a single-agent, but immune checkpoint inhibitors (ICI) offer additional options, particularly antibodies targeting programmed cell death 1 protein (PD-1) or its ligand PD-L1. The observed objective response rates (ORR) for ICI therapies ranged from 15-21% in unselected population.

Thus, there exists a need for biomarkers to identify a specific group of patients suffering from urothelial carcinoma who are most likely to respond to the treatment of ICI.

### SUMMARY OF THE INVENTION

In one aspect, the present invention provides a method of treating a patient suffering from urothelial carcinoma, comprising determining a tumor mutational burden of the patient: identifying a candidate exhibiting a high tumor mutational burden, wherein the high tumor mutational burden is ≥10 mutations/Mbp; and administering to the candidate a therapeutically effective amount of an inhibitor selected from anti-PD-1 antibody. In one embodiment, the urothelial carcinoma is locally advanced or metastatic urothelial carcinoma. In another embodiment, the inhibitor is toripalimab.

In another aspect, the present invention provides a method of treating a patient suffering from urothelial carcinoma, comprising identifying a candidate having mutations in one or more of the following genes occurred in tumor cells: SMARCA4 and RB1; and administering to the candidate a therapeutically effective amount of an inhibitor selected from anti-PD-1 antibody. In one embodiment, the urothelial carcinoma is locally advanced or metastatic urothelial carcinoma. In another embodiment, the inhibitor is toripalimab.

In another aspect, the present invention provides a method of predicting the response of a patient suffering from urothelial carcinoma to the treatment comprising a therapeutically effective amount of an inhibitor selected from anti-PD-1 antibody, comprising determining a tumor mutational burden of the patient: comparing the tumor mutational burden with a predetermined reference value, wherein the predetermined reference value is ≥10 mutations/Mbp: concluding that the patient is more likely to respond to the treatment if tumor mutational burden is equal with or higher than the predetermined reference value, compared with the corresponding control group. In one embodiment, the urothelial carcinoma is locally advanced or metastatic urothelial carcinoma. In another embodiment, the inhibitor is toripalimab.

In another aspect, the present invention provides a method of predicting the response of a patient suffering from urothelial carcinoma to the treatment comprising a therapeutically effective amount of an inhibitor selected from anti-PD-1 antibody, comprising identifying a patient having mutations in one or more of the following genes occurred in tumor cells: SMARCA4 and RB1; and concluding that the patient is more likely to respond to the treatment, compared with the corresponding control group. In one embodiment, the urothelial carcinoma is locally advanced or metastatic urothelial carcinoma. In another embodiment, the inhibitor is toripalimab.

In one aspect, the present invention provides use of a composition comprising an inhibitor selected from anti-PD-1 antibody in the manufacture of a medicament for treating a patient suffering from urothelial carcinoma, wherein the patient exhibits a high tumor mutational burden of ≥10 mutations/Mbp. In one embodiment, the urothelial carcinoma is locally advanced or metastatic urothelial carcinoma. In another embodiment, the inhibitor is toripalimab.

In another aspect, the present invention provides use of a composition comprising an inhibitor selected from anti-PD-1 antibody in the manufacture of a medicament for treating a patient suffering from urothelial carcinoma, wherein the patient has mutations in one or more of the following genes occurred in tumor cells: SMARCA4 and RB1. In one embodiment, the urothelial carcinoma is locally advanced or metastatic urothelial carcinoma. In another embodiment, the inhibitor is toripalimab.

In another aspect, the present invention provides use of a reagent for determining a tumor mutational burden in the manufacture of a medicament for predicting the response of the patient suffering from urothelial carcinoma to the treatment comprising a therapeutically effective amount of an inhibitor selected from anti-PD-1 antibody, wherein the predicting comprises determining a tumor mutational burden of the patient; comparing the tumor mutational burden with a predetermined reference value, wherein the predetermined reference value is ≥10 mutations/Mbp: concluding that the patient is more likely to respond to the treatment if tumor mutational burden is equal with or higher than the predetermined reference value, compared with the corresponding control group. In one embodiment, the urothelial carcinoma is locally advanced or metastatic urothelial carcinoma. In another embodiment, the inhibitor is toripalimab.

In another aspect, the present invention provides use of a reagent for determining mutations in the manufacture of a medicament for predicting the response of the patient suffering from urothelial carcinoma to the treatment comprising a therapeutically effective amount of an inhibitor selected from anti-PD-1 antibody, wherein the predicting comprises identifying a patient having mutations in one or more of the following genes occurred in tumor cells: SMARCA4 and RB1; and concluding that the patient is more likely to respond to the treatment, compared with the corresponding control group. In one embodiment, the urothelial carcinoma is locally advanced or metastatic urothelial carcinoma. In another embodiment, the inhibitor is toripalimab.

In one aspect, the present invention provides a composition comprising an inhibitor selected from anti-PD-1 antibody for use in the treatment of a patient suffering from urothelial carcinoma, wherein the patient exhibits a high tumor mutational burden of ≥10) mutations/Mbp. In one embodiment, the urothelial carcinoma is locally advanced or metastatic urothelial carcinoma. In another embodiment, the inhibitor is toripalimab.

In another aspect, the present invention provides a composition comprising an inhibitor selected from anti-PD-1 antibody for use in the treatment of a patient suffering from urothelial carcinoma, wherein the patient has mutations in one or more of the following genes occurred in tumor cells: SMARCA4 and RB1. In one embodiment, the urothelial carcinoma is locally advanced or metastatic urothelial carcinoma. In another embodiment, the inhibitor is toripalimab.

In another aspect, the present invention provides a reagent for determining a tumor mutational burden for use in predicting the response of the patient suffering from urothelial carcinoma to the treatment comprising a therapeutically effective amount of an inhibitor selected from anti-PD-1 antibody, wherein the predicting comprises determining a tumor mutational burden of the patient: comparing the tumor mutational burden with a predetermined reference value, wherein the predetermined reference value is ≥10 mutations/Mbp: concluding that the patient is more likely to respond to the treatment if tumor mutational burden is equal with or higher than the predetermined reference value, compared with the corresponding control group. In one embodiment, the urothelial carcinoma is locally advanced or metastatic urothelial carcinoma. In another embodiment, the inhibitor is toripalimab.

In another aspect, the present invention provides a reagent for determining mutations for use in predicting the response of the patient suffering from urothelial carcinoma to the treatment comprising a therapeutically effective amount of an inhibitor selected from anti-PD-1 antibody, wherein the predicting comprises identifying a patient having mutations in one or more of the following genes occurred in tumor cells: SMARCA4 and RB1; and concluding that the patient is more likely to respond to the treatment, compared with the corresponding control group. In one embodiment, the urothelial carcinoma is locally advanced or metastatic urothelial carcinoma. In another embodiment, the inhibitor is toripalimab.

In one aspect, the present invention provides a composition comprising an inhibitor selected from anti-PD-1 antibody as the effective ingredient for treating a patient suffering from urothelial carcinoma, wherein the patient exhibits a high tumor mutational burden of ≥10 mutations/Mbp. In one embodiment, the urothelial carcinoma is locally advanced or metastatic urothelial carcinoma. In another embodiment, the inhibitor is toripalimab.

In another aspect, the present invention provides a composition comprising an inhibitor selected from anti-PD-1 antibody as the effective ingredient for treating a patient suffering from urothelial carcinoma, wherein the patient has mutations in one or more of the following genes occurred in tumor cells: SMARCA4 and RB1. In one embodiment, the urothelial carcinoma is locally advanced or metastatic urothelial carcinoma. In another embodiment, the inhibitor is toripalimab.

In another aspect, the present invention provides a composition comprising a reagent for determining a tumor mutational burden as the effective ingredient for predicting the response of the patient suffering from urothelial carcinoma to the treatment comprising a therapeutically effective amount of an inhibitor selected from anti-PD-1 antibody, wherein the predicting comprises determining a tumor mutational burden of the patient: comparing the tumor mutational burden with a predetermined reference value, wherein the predetermined reference value is ≥10 mutations/Mbp; concluding that the patient is more likely to respond to the treatment if tumor mutational burden is equal with or higher than the predetermined reference value, compared with the corresponding control group. In one embodiment, the urothelial carcinoma is locally advanced or metastatic urothelial carcinoma. In another embodiment, the inhibitor is toripalimab.

In another aspect, the present invention provides a composition comprising a reagent for determining mutations as the effective ingredient for predicting the response of the patient suffering from urothelial carcinoma to the treatment comprising a therapeutically effective amount of an inhibitor selected from anti-PD-1 antibody, wherein the predicting comprises identifying a patient having mutations in one or more of the following genes occurred in tumor cells: SMARCA4 and RB1; and concluding that the patient is more likely to respond to the treatment, compared with the corresponding control group. In one embodiment, the urothelial carcinoma is locally advanced or metastatic urothelial carcinoma. In another embodiment, the inhibitor is toripalimab.

### BRIEF DESCRIPTION OF THE DRAWINGS

The following figures are presented for the purpose of illustration only, and are not intended to be limiting.

FIG. 1 shows cross correlation study among different PD-L1 IHC staining antibody including JS311, SP263, SP142, and 22C3 in tumor biopsies from three cancer types. JS311 showed similar PD-L1 staining patterns and scores with SP263 antibody. PD-L1 positive was defined as tumor proportion score (TPS) ≥1%, namely the presence of membrane staining of any intensity in ≥1% of tumor cells (TC). Tumor biopsies: 1-10 non-small cell lung cancer: 11-20 melanoma: 21-30 urothelial carcinoma.

FIG. 2 shows CONSORT diagram for the Phase II study of toripalimab in patients with locally advanced or metastatic urothelial carcinoma after failure of standard therapy.

FIG. 3 (A) Maximal change of tumor size from baseline assessed by IRC per RECIST v1.1. The length of the bar represents maximal decrease or minimal increase in target lesion(s). PD-L1 positive status was defined as the presence of membrane staining of any intensity in ≥1% of tumor cells by JS311 IHC staining. Tumor mutational burden (TMB) was determined by whole exome sequencing. (B) Change of individual tumor burden over time from baseline assessed by IRC per RECIST v1.1. (C) Exposure and duration of response per RECIST v1.1.

FIG. 4 (A) Progression-free survival and (B) overall survival of all patients (n=151) in the study. (C) Duration of response of responding patients (n=39) in the study. Percentages of survival patients are shown at indicated time points. Censored patients are marked with “|” in the graph. Numbers of patients at risk at indicated time points are shown below the x-axis.

FIG. 5 (A) Clinical response in relation to tumor PD-L1 expression and tumor mutational burden. PD-L1 positive status was defined as the presence of membrane staining of any intensity in ≥1% of tumor cells by JS311 IHC staining. The tumor mutational burden (TMB) was calculated by total somatic mutations within the coding regions by whole exome sequencing. Number of PD-L1+, PD-L1−, TMB^{high} (TMB≥10 mutations/Mbp) and TMB^{low} (TMB<10 mutations/Mbp) subject are shown in the bottom table. (B) Progression-free survival of PD-L1+ versus PD-L1− patients. (C) Overall survival of PD-L1+ versus PD-L1− patients. (D) Progression-free survival of TMB≥10 Muts/Mb versus TMB<10 Muts/Mb patients. (E) Overall survival of TMB≥10 Muts/Mb versus TMB<10 Muts/Mb patients. PD-L1 positive status was defined as the presence of membrane staining of any intensity in ≥1% of tumor cells by JS311 IHC staining. Percentages of survival patients are shown at indicated time points. Censored patients are marked with “| ” in the graph. Numbers of patients at risk at indicated time points are shown below the x-axis. NE, not estimable.

FIG. 6 Genetic alternations and frequencies identified by whole exome sequencing (WES) from 135 available patients. Patients were grouped by clinical responses.

### DETAILED DESCRIPTION OF THE INVENTION

The present study is the largest to date to investigate the safety and anti-tumor activity of a PD-1 antibody in the second line setting for patients with mUC with whole exome sequencing (WES) and tumor mutational burden (TMB) analysis.

Toripalimab monotherapy provided a confirmed objective response rate (ORR) of 25.8%, progression free survival (PFS) of 2.3 months, overall survival (OS) of 14.4 months in the intent to treat population and an objective response rate (ORR) of 41.7%, progression free survival (PFS) of 3.7 months, overall survival (OS) of 35.6 months in the PD-L1+ patients.

Surprisingly, patients with tumor mutational burden (TMB) ≥10 mutations per million base pairs exhibited the confirmed objective response rate (ORR) of 48.1%, progression free survival (PFS) of 12.9 months, overall survival (OS) not reached, significantly higher than unspecified group. Furthermore, the TMB high group showed significantly better ORR (48.1% v. 22.2%), PFS (median PFS 12.9 versus 1.8 months) and OS (median OS not reached vs 10.0 months) than the TMB low group.

Patients having mutations in chromatin remodeler SMARCA4 or tumor suppressor RB1 exhibited significantly better response to toripalimab with an ORR of 58.3% versus 24.4% for wild type.

Lymph node only metastasis had significantly better ORR than patients with visceral metastasis, 52.6% versus 22.0%.

To the best of our knowledge, this is the first prospective clinical trial demonstrating a response rate greater than 40% for biomarker selected 2nd line metastatic urothelial carcinoma receiving immune checkpoint inhibitors (ICI) therapy. It is reported, for the first time, the utility of biomarkers such as tumor mutational burden (TMB) in patients with metastatic urothelial carcinoma to predict not only the ORR but also PFS and OS benefits in response to immune checkpoint inhibitors (ICI) therapy.

The biomarker for predicting the response of a patient suffering from urothelial carcinoma to toripalimab comprises any one of the following biomarks: tumor mutational burden (TMB) ≥10 mutations per million base pairs: genomic mutations in SMARCA4: genomic mutations in RB1: lymph node only metastasis; and positive PD-L1 expression in a tumor sample, and the combination thereof.

Second-line treatment with toripalimab for mUC showed a clinical meaningful anti-tumor activity with a manageable safety profile. The observed objective response rates were the highest in both unselected and PD-L1+ patients among the class of immune checkpoint inhibitors (ICI) drugs. The patients having one or more of the biomarkers selected from the group consisting of tumor mutational burden (TMB) ≥10 mutations per million base pairs: genomic mutations in SMARCA4: genomic mutations in RB1: lymph node only metastasis; and positive PD-L1 expression in a tumor sample exhibit better response, such as increased ORR, PFS, OS, etc. In order to further enhance the effect of the treatment, any one or any combination of biomarkers selected from the group consisting of tumor mutational burden (TMB) ≥10 mutations per million base pairs: genomic mutations in SMARCA4: genomic mutations in RB1: lymph node only metastasis; and positive PD-L1 expression in a tumor sample could be used to identify mUC patients who are most likely to benefit from ICI monotherapy (such as toripalimab) in the second-line setting.

In one aspect, the present invention provides a method of treating a patient suffering from urothelial carcinoma, comprising determining a tumor mutational burden of the patient: identifying a candidate exhibiting a high tumor mutational burden, wherein the high tumor mutational burden is ≥10 mutations/Mbp; and administering to the candidate a therapeutically effective amount of an inhibitor selected from anti-PD-1 antibody.

In one embodiment, the urothelial carcinoma is locally advanced or metastatic urothelial carcinoma. In another embodiment, the patient has received a treatment of chemotherapy.

In one embodiment, the tumor mutational burden is determined by performing whole exome sequencing. In another embodiment, the tumor mutational burden is determined by analyzing genomic mutations selected from the group consisting of microsatellite stability status, single base substitution, short and long insertions/deletions, copy number variants, and gene rearrangement and fusions, wherein the genomic mutations are somatice mutations. In another embodiment, at least one of the genomic mutations occur in one or more of the following genes: TP53, TERT, KMT2D, CDKN2A, CDKN2B, KDM2A, ERBB2, MTAP, ARID1A, CCND1, FGF19, PIK3CA, FGF4, FGF3, FGFR3, CREBBP, E2F3, KMT2C, NOTCH1, ATM1 and NECTIN4. In another embodiment, the candidate is further identified as having genomic mutations in one or more of the following genes: SMARCA4 and RB1. In another embodiment, the candidate is further identified as having genomic mutations in FGFR2 and/or FGFR3, optionally, in FGFR3 gene mutation or FGFR2/FGFR3 gene fusion. In another embodiment, the method further comprises administering to the candidate erdafitinib. In another embodiment, the candidate is further identified as having genomic mutations in NECTIN4, optionally, in NECTIN4 gene amplification. In another embodiment, the method further comprises administering to the candidate enfortumab vedotin.

In one embodiment, the candidate is further identified as exhibiting positive PD-L1 expression in a tumor sample. In another embodiment, the candidate is further identified as having lymph node only metastasis.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In another aspect, the present invention provides a method of treating a patient suffering from urothelial carcinoma, comprising identifying a candidate having mutations in one or more of the following genes occurred in tumor cells: SMARCA4 and RB1; and administering to the candidate a therapeutically effective amount of an inhibitor selected from anti-PD-1 antibody.

In one embodiment, the urothelial carcinoma is locally advanced or metastatic urothelial carcinoma. In another embodiment, the patient has received a treatment of chemotherapy. In one embodiment, the mutations are somatic mutations.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In another aspect, the present invention provides a method of predicting the response of a patient suffering from urothelial carcinoma to the treatment comprising a therapeutically effective amount of an inhibitor selected from anti-PD-1 antibody, comprising determining a tumor mutational burden of the patient: comparing the tumor mutational burden with a predetermined reference value, wherein the predetermined reference value is ≥10 mutations/Mbp: concluding that the patient is more likely to respond to the treatment if tumor mutational burden is equal with or higher than the predetermined reference value, compared with the corresponding control group.

In one embodiment, the urothelial carcinoma is locally advanced or metastatic urothelial carcinoma. In another embodiment, the patient has received a treatment of chemotherapy.

In one embodiment, the tumor mutational burden is determined by performing whole exome sequencing. In another embodiment, the tumor mutational burden is determined by analyzing genomic mutations selected from the group consisting of microsatellite stability status, single base substitution, short and long insertions/deletions, copy number variants, and gene rearrangement and fusions, wherein the genomic mutations are somatice mutations. In another embodiment, at least one of the genomic mutations occur in one or more of the following genes: TP53, TERT, KMT2D, CDKN2A, CDKN2B, KDM2A, ERBB2, MTAP, ARID1A, CCND1, FGF19, PIK3CA, FGF4, FGF3, FGFR3, CREBBP, E2F3, KMT2C, NOTCH1, ATM1 and NECTIN4. In another embodiment, the patient further has genomic mutations in one or more of the following genes: SMARCA4 and RB1.

In one embodiment, the patient further exhibits positive PD-L1 expression in a tumor sample. In another embodiment, the patient further has lymph node only metastasis.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In another aspect, the present invention provides a method of predicting the response of a patient suffering from urothelial carcinoma to the treatment comprising a therapeutically effective amount of an inhibitor selected from anti-PD-1 antibody, comprising identifying a patient having mutations in one or more of the following genes occurred in tumor cells: SMARCA4 and RB1; and concluding that the patient is more likely to respond to the treatment, compared with the corresponding control group.

In one embodiment, the urothelial carcinoma is locally advanced or metastatic urothelial carcinoma. In another embodiment, the patient has received a treatment of chemotherapy. In another embodiment, the mutations are somatic mutations.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In one aspect, the present invention provides use of a composition comprising an inhibitor selected from anti-PD-1 antibody in the manufacture of a medicament for treating a patient suffering from urothelial carcinoma, wherein the patient exhibits a high tumor mutational burden of ≥10 mutations/Mbp.

In one embodiment, the urothelial carcinoma is locally advanced or metastatic urothelial carcinoma. In another embodiment, the patient has received a treatment of chemotherapy.

In one embodiment, the tumor mutational burden is determined by performing whole exome sequencing. In another embodiment, the tumor mutational burden is determined by analyzing genomic mutations selected from the group consisting of microsatellite stability status, single base substitution, short and long insertions/deletions, copy number variants, and gene rearrangement and fusions, wherein the genomic mutations are somatice mutations. In another embodiment, at least one of the genomic mutations occur in one or more of the following genes: TP53, TERT, KMT2D, CDKN2A, CDKN2B, KDM2A, ERBB2, MTAP, ARID1A, CCND1, FGF19, PIK3CA, FGF4, FGF3, FGFR3, CREBBP, E2F3, KMT2C, NOTCH1, ATM1 and NECTIN4. In another embodiment, the patient further has genomic mutations in one or more of the following genes: SMARCA4 and RB1. In another embodiment, the patient further has genomic mutations in FGFR2 and/or FGFR3, optionally, in FGFR3 gene mutation or FGFR2/FGFR3 gene fusion. In another embodiment, the composition further comprises erdafitinib. In another embodiment, the patient further has genomic mutations in NECTIN4, optionally, in NECTIN4 gene amplification. In another embodiment, the composition further comprises enfortumab vedotin.

In one embodiment, the patient further exhibits positive PD-L1 expression in a tumor sample. In another embodiment, the patient further has lymph node only metastasis.

In one embodiment, the inhibitor is an anti-PD-1/PD-L antibody. In another embodiment, the inhibitor is pembrolizumab, nivolumab, tislelizumab, sintilimab, camrelizumab, or cemiplimab. In another embodiment, the inhibitor is toripalimab.

In another aspect, the present invention provides use of a composition comprising an inhibitor selected from anti-PD-1 antibody in the manufacture of a medicament for treating a patient suffering from urothelial carcinoma, wherein the patient has mutations in one or more of the following genes occurred in tumor cells: SMARCA4 and RB1.

In one embodiment, the urothelial carcinoma is locally advanced or metastatic urothelial carcinoma. In another embodiment, the patient has received a treatment of chemotherapy. In one embodiment, the mutations are somatic mutations.

In one embodiment, the inhibitor is an anti-PD-1 antibody. In one embodiment, the inhibitor is pembrolizumab, nivolumab, tislelizumab, sintilimab, camrelizumab, or cemiplimab. In another embodiment, the inhibitor is toripalimab.

In another aspect, the present invention provides use of a reagent for determining a tumor mutational burden in the manufacture of a medicament for predicting the response of the patient suffering from urothelial carcinoma to the treatment comprising a therapeutically effective amount of an inhibitor selected from anti-PD-1 antibody, wherein the predicting comprises determining a tumor mutational burden of the patient; comparing the tumor mutational burden with a predetermined reference value, wherein the predetermined reference value is ≥10 mutations/Mbp: concluding that the patient is more likely to respond to the treatment if tumor mutational burden is equal with or higher than the predetermined reference value, compared with the corresponding control group.

In one embodiment, the urothelial carcinoma is locally advanced or metastatic urothelial carcinoma. In another embodiment, the patient has received a treatment of chemotherapy.

In one embodiment, the tumor mutational burden is determined by performing whole exome sequencing. In another embodiment, the tumor mutational burden is determined by analyzing genomic mutations selected from the group consisting of microsatellite stability status, single base substitution, short and long insertions/deletions, copy number variants, and gene rearrangement and fusions, wherein the genomic mutations are somatice mutations. In another embodiment, at least one of the genomic mutations occur in one or more of the following genes: TP53, TERT, KMT2D, CDKN2A, CDKN2B, KDM2A, ERBB2, MTAP, ARID1A, CCND1, FGF19, PIK3CA, FGF4, FGF3, FGFR3, CREBBP, E2F3, KMT2C, NOTCH1, ATM1 and NECTIN4. In another embodiment, the patient further has genomic mutations in one or more of the following genes: SMARCA4 and RB1.

In one embodiment, the patient further exhibits positive PD-L1 expression in a tumor sample. In another embodiment, the patient further has lymph node only metastasis.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In another aspect, the present invention provides use of a reagent for determining mutations in the manufacture of a medicament for predicting the response of the patient suffering from urothelial carcinoma to the treatment comprising a therapeutically effective amount of an inhibitor selected from anti-PD-1 antibody, wherein the predicting comprises identifying a patient having mutations in one or more of the following genes occurred in tumor cells: SMARCA4 and RB1; and concluding that the patient is more likely to respond to the treatment, compared with the corresponding control group.

In one embodiment, the urothelial carcinoma is locally advanced or metastatic urothelial carcinoma. In another embodiment, the patient has received a treatment of chemotherapy. In another embodiment, the mutations are somatic mutations.

In one embodiment, the inhibitor is an anti-PD-1 antibody. In one embodiment, the inhibitor is pembrolizumab, nivolumab, tislelizumab, sintilimab, camrelizumab, or cemiplimab. In one embodiment, the inhibitor is toripalimab.

In one aspect, the present invention provides a composition comprising an inhibitor selected from anti-PD-1 antibody for use in the treatment of a patient suffering from urothelial carcinoma, wherein the patient exhibits a high tumor mutational burden of ≥10 mutations/Mbp.

In one embodiment, the urothelial carcinoma is locally advanced or metastatic urothelial carcinoma. In another embodiment, the patient has received a treatment of chemotherapy.

In one embodiment, the tumor mutational burden is determined by performing whole exome sequencing. In another embodiment, the tumor mutational burden is determined by analyzing genomic mutations selected from the group consisting of microsatellite stability status, single base substitution, short and long insertions/deletions, copy number variants, and gene rearrangement and fusions, wherein the genomic mutations are somatice mutations. In another embodiment, at least one of the genomic mutations occur in one or more of the following genes: TP53, TERT, KMT2D, CDKN2A, CDKN2B, KDM2A, ERBB2, MTAP, ARID1A, CCND1, FGF19, PIK3CA, FGF4, FGF3, FGFR3, CREBBP, E2F3, KMT2C, NOTCH1, ATM1 and NECTIN4. In another embodiment, the patient further has genomic mutations in one or more of the following genes: SMARCA4 and RB1. In one embodiment, the patient further has genomic mutations in FGFR2 and/or FGFR3, optionally, in FGFR3 gene mutation or FGFR2/FGFR3 gene fusion. In another embodiment, the composition further comprises erdafitinib. In another embodiment, the patient further has genomic mutations in NECTIN4, optionally, in NECTIN4 gene amplification. In another embodiment, the composition further comprises enfortumab vedotin.

In one embodiment, the patient further exhibits positive PD-L1 expression in a tumor sample. In one embodiment, the patient further has lymph node only metastasis.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In another aspect, the present invention provides a composition comprising an inhibitor selected from anti-PD-1 antibody for use in the treatment of a patient suffering from urothelial carcinoma, wherein the patient has mutations in one or more of the following genes occurred in tumor cells: SMARCA4 and RB1.

In one embodiment, the urothelial carcinoma is locally advanced or metastatic urothelial carcinoma. In another embodiment, the patient has received a treatment of chemotherapy. In another embodiment, the mutations are somatic mutations.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In another aspect, the present invention provides a reagent for determining a tumor mutational burden for use in predicting the response of the patient suffering from urothelial carcinoma to the treatment comprising a therapeutically effective amount of an inhibitor selected from anti-PD-1 antibody, wherein the predicting comprises determining a tumor mutational burden of the patient: comparing the tumor mutational burden with a predetermined reference value, wherein the predetermined reference value is ≥10 mutations/Mbp: concluding that the patient is more likely to respond to the treatment if tumor mutational burden is equal with or higher than the predetermined reference value, compared with the corresponding control group.

In one embodiment, the urothelial carcinoma is locally advanced or metastatic urothelial carcinoma. In another embodiment, the patient has received a treatment of chemotherapy.

In one embodiment, the tumor mutational burden is determined by performing whole exome sequencing. In another embodiment, wherein the tumor mutational burden is determined by analyzing genomic mutations selected from the group consisting of microsatellite stability status, single base substitution, short and long insertions/deletions, copy number variants, and gene rearrangement and fusions, wherein the genomic mutations are somatice mutations. In another embodiment, at least one of the genomic mutations occur in one or more of the following genes: TP53, TERT. KMT2D, CDKN2A, CDKN2B, KDM2A, ERBB2, MTAP, ARID1A, CCND1, FGF19, PIK3CA, FGF4, FGF3, FGFR3, CREBBP, E2F3, KMT2C, NOTCH1, ATM1 and NECTIN4. In another embodiment, the patient further has genomic mutations in one or more of the following genes: SMARCA4 and RB1.

In one embodiment, the patient further exhibits positive PD-L1 expression in a tumor sample. In another embodiment, the patient further has lymph node only metastasis.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In another aspect, the present invention provides a reagent for determining mutations for use in predicting the response of the patient suffering from urothelial carcinoma to the treatment comprising a therapeutically effective amount of an inhibitor selected from anti-PD-1 antibody, wherein the predicting comprises identifying a patient having mutations in one or more of the following genes occurred in tumor cells: SMARCA4 and RB1; and concluding that the patient is more likely to respond to the treatment, compared with the corresponding control group.

In one embodiment, the urothelial carcinoma is locally advanced or metastatic urothelial carcinoma. In another embodiment, the patient has received a treatment of chemotherapy. In another embodiment, the mutations are somatic mutations.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In one aspect, the present invention provides a composition comprising an inhibitor selected from anti-PD-1 antibody as the effective ingredient for treating a patient suffering from urothelial carcinoma, wherein the patient exhibits a high tumor mutational burden of ≥10 mutations/Mbp.

In one embodiment, the urothelial carcinoma is locally advanced or metastatic urothelial carcinoma. In another embodiment, the patient has received a treatment of chemotherapy.

In one embodiment, the tumor mutational burden is determined by performing whole exome sequencing. In another embodiment, the tumor mutational burden is determined by analyzing genomic mutations selected from the group consisting of microsatellite stability status, single base substitution, short and long insertions/deletions, copy number variants, and gene rearrangement and fusions, wherein the genomic mutations are somatice mutations. In another embodiment, at least one of the genomic mutations occur in one or more of the following genes: TP53, TERT, KMT2D, CDKN2A, CDKN2B, KDM2A, ERBB2, MTAP, ARID1A, CCND1, FGF19, PIK3CA, FGF4, FGF3, FGFR3, CREBBP, E2F3, KMT2C, NOTCH1, ATM1 and NECTIN4. In another embodiment, the patient further has genomic mutations in one or more of the following genes: SMARCA4 and RB1. In another embodiment, the patient further has genomic mutations in FGFR2 and/or FGFR3, optionally, in FGFR3 gene mutation or FGFR2/FGFR3 gene fusion. In another embodiment, the composition further comprises erdafitinib. In another embodiment, the patient further has genomic mutations in NECTIN4, optionally, in NECTIN4 gene amplification. In another embodiment, the composition further comprises enfortumab vedotin.

In one embodiment, the patient further exhibits positive PD-L1 expression in a tumor sample. In another embodiment, the patient further has lymph node only metastasis.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In another aspect, the present invention provides a composition comprising an inhibitor selected from anti-PD-1 antibody as the effective ingredient for treating a patient suffering from urothelial carcinoma, wherein the patient has mutations in one or more of the following genes occurred in tumor cells: SMARCA4 and RB1.

In one embodiment, the urothelial carcinoma is locally advanced or metastatic urothelial carcinoma. In another embodiment, the patient has received a treatment of chemotherapy. In another embodiment, the mutations are somatic mutations.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In another aspect, the present invention provides a composition comprising a reagent for determining a tumor mutational burden as the effective ingredient for predicting the response of the patient suffering from urothelial carcinoma to the treatment comprising a therapeutically effective amount of an inhibitor selected from anti-PD-1 antibody, wherein the predicting comprises determining a tumor mutational burden of the patient: comparing the tumor mutational burden with a predetermined reference value, wherein the predetermined reference value is ≥10 mutations/Mbp; concluding that the patient is more likely to respond to the treatment if tumor mutational burden is equal with or higher than the predetermined reference value, compared with the corresponding control group.

In one embodiment, the urothelial carcinoma is locally advanced or metastatic urothelial carcinoma. In another embodiment, the patient has received a treatment of chemotherapy.

In one embodiment, the tumor mutational burden is determined by performing whole exome sequencing. In another embodiment, the tumor mutational burden is determined by analyzing genomic mutations selected from the group consisting of microsatellite stability status, single base substitution, short and long insertions/deletions, copy number variants, and gene rearrangement and fusions, wherein the genomic mutations are somatice mutations. In another embodiment, at least one of the genomic mutations occur in one or more of the following genes: TP53, TERT, KMT2D, CDKN2A, CDKN2B, KDM2A, ERBB2, MTAP, ARID1A, CCND1, FGF19, PIK3CA, FGF4, FGF3, FGFR3, CREBBP, E2F3, KMT2C, NOTCH1, ATM1 and NECTIN4. In another embodiment, the patient has genomic mutations in one or more of the following genes: SMARCA4 and RB1.

In one embodiment, the patient further exhibits positive PD-L1 expression in a tumor sample. In another embodiment, the patient further has lymph node only metastasis.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In another aspect, the present invention provides a composition comprising a reagent for determining mutations as the effective ingredient for predicting the response of the patient suffering from urothelial carcinoma to the treatment comprising a therapeutically effective amount of an inhibitor selected from anti-PD-1 antibody, wherein the predicting comprises identifying a patient having mutations in one or more of the following genes occurred in tumor cells: SMARCA4 and RB1; and concluding that the patient is more likely to respond to the treatment, compared with the corresponding control group.

In one embodiment, the urothelial carcinoma is locally advanced or metastatic urothelial carcinoma. In another embodiment, the patient has received a treatment of chemotherapy. In another embodiment, the mutations are somatic mutations.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

In one aspect, the present invention provides use of a composition comprising toripalimab for treating a patient suffering from urothelial carcinoma, wherein the patient exhibits a high tumor mutational burden of ≥10 mutations/Mbp.

In another aspect, the present invention provides use of a composition comprising toripalimab for treating a patient suffering from urothelial carcinoma, wherein the patient has mutations in one or more of the following genes occurred in tumor cells: SMARCA4 and RB1.

In another aspect, the present invention provides use of a reagent for determining a tumor mutational burden for predicting the response of the patient suffering from urothelial carcinoma to the treatment comprising a therapeutically effective amount of toripalimab, wherein the predicting comprises determining a tumor mutational burden of the patient: comparing the tumor mutational burden with a predetermined reference value, wherein the predetermined reference value is ≥10 mutations/Mbp: concluding that the patient is more likely to respond to the treatment if tumor mutational burden is equal with or higher than the predetermined reference value, compared with the corresponding control group.

In another aspect, the present invention provides use of a reagent for determining mutations for predicting the response of the patient suffering from urothelial carcinoma to the treatment comprising a therapeutically effective amount of toripalimab, wherein the predicting comprises identifying a patient having mutations in one or more of the following genes occurred in tumor cells: SMARCA4 and RB1; and concluding that the patient is more likely to respond to the treatment, compared with the corresponding control group.

### (I) Method of Treating and Method of Predicting

In a first aspect, the present invention provides a method of treating a patient suffering from urothelial carcinoma, comprising determining a tumor mutational burden of the patient: identifying a candidate exhibiting a high tumor mutational burden, wherein the high tumor mutational burden is ≥10 mutations/Mbp; and administering to the candidate a therapeutically effective amount of an inhibitor selected from anti-PD-1 antibody.

In one embodiment, the high tumor mutational burden is ≥6, 7, 8 or 9 mutations/Mbp.

In one embodiment, the urothelial carcinoma is locally advanced or metastatic urothelial carcinoma. In another embodiment, the urothelial carcinoma is non-metastatic urothelial carcinoma. In one embodiment, the urothelial carcinoma is lower tract urothelial carcinoma (LTUC) originated from bladder or urethral canal. In another embodiment, the urothelial carcinoma is upper tract urothelial carcinoma (UTUC) from renal pelvis or ureter. In one embodiment, the tumor metastasis is lymph node only. In another embodiment, the tumor metastasis is visceral.

In one embodiment, the patient has received a treatment of chemotherapy. In one embodiment, the patient has received a first-line treatment of chemotherapy. In another embodiment, the patient has not received a first-line treatment of chemotherapy before. In another embodiment, the patient has received two lines of chemotherapy. In one embodiment, the patient has received a platinum-based chemotherapy. In another embodiment, the patient has received a non-platinum chemotherapy. In one embodiment, the patient has failed in the previous standard chemotherapy.

In one embodiment, the tumor mutational burden is determined by performing whole exome sequencing. In another embodiment, the tumor mutational burden is determined by performing whole genome sequencing. In one embodiment, the whole exome sequencing or whole genome sequencing is performed on tumor samples, such as tumor biopsies.

In one embodiment, the tumor mutational burden is determined by analyzing genomic mutations, including microsatellite stability status, single base substitution, short and long insertions/deletions, copy number variants, and gene rearrangement and fusions, missense mutations, frameshift mutations, nonsense mutation, duplications and repeat expansions. In another embodiment, the genomic mutations are somatic mutations. In another embodiment, the genomic mutations are somatic mutations within the coding regions. In another embodiment, the genomic mutations are somatic mutations within the coding regions and non-coding regions. In one embodiment, the tumor mutational burden is determined by analyzing genomic mutations selected from the group consisting of microsatellite stability status, single base substitution, short and long insertions/deletions, copy number variants, and gene rearrangement and fusions, wherein the genomic mutations are somatic mutations (optionally within coding regions).

In one embodiment, at least one of the genomic mutations occur in one or more of the following genes: TP53, TERT, KMT2D, CDKN2A, CDKN2B, KDM2A, ERBB2, MTAP, ARID1A, CCND1, FGF19, PIK3CA, FGF4, FGF3, FGFR3, CREBBP, E2F3, KMT2C, NOTCH1, ATM1 and NECTIN4.

In one embodiment, the candidate is further identified as having genomic mutations in FGFR2 and/or FGFR3, preferably, in FGFR3 gene mutation or FGFR2/FGFR3 gene fusion. Under this circumstances, the combination of an inhibitor selected from anti-PD-1 antibody (such as toripalimab) and erdafitinib is administered to the candidate. In another embodiment, the candidate is further identified as having genomic mutations in NECTIN4, optionally, in NECTIN4 gene amplification. Under this circumstances, the combination of an inhibitor selected from anti-PD-1 antibody (such as toripalimab) and enfortumab vedotin is administered to the candidate. In recent years, additional targeting therapies for later line treatment of mUC have been approved by the US FDA, including erdafitinib for patients with certain FGFR3 gene mutations or FGFR2/FGFR3 gene fusions and enfortumab vedotin for nectin-4 positive mUC.

In one embodiment, the patient is further identified as having genomic mutations in one or more of the following genes: SMARCA4 and RB1. In one embodiment, the patient is further identified as having genomic mutations in SMARCA4. In another embodiment, the patient is further identified as having genomic mutations in RB1. In another embodiment, the patient is further identified as having genomic mutations in SMARCA4 and RB1. Patients with TMB high (≥10 mutations/Mb) in combination with genomic mutations in one or more of the following genes: SMARCA4 and RB1 (optionally, somatic mutations in tumor cells) showed significantly better response (such as ORR, PFS, OS, etc.) to toripalimab than patients with either one biomarker alone or both biomarkers. TMB high (≥10) mutations/Mb) in combination with genomic mutations in one or more of the following genes: SMARCA4 and RB1 (optionally, somatic mutations in tumor cells) could be used to identify mUC patients who are most likely to benefit from an inhibitor selected from anti-PD-1 antibody (such as toripalimab).

In one embodiment, the patient is identified as having lymph node only metastasis. Patients having lymph node only metastasis exhibit significantly better response (such as ORR, PFS, OS, etc.) to toripalimab than patients with visceral metastasis. In one aspect, the present invention provides a method of treating a patient suffering from urothelial carcinoma, comprising identifying a candidate having lymph node only metastasis; and administering to the candidate a therapeutically effective amount of an inhibitor selected from anti-PD-1 antibody, such as toripalimab.

In one embodiment, the patient is further identified as having lymph node only metastasis. Patients with TMB high (≥10 mutations/Mb) and lymph node only metastasis showed significantly better response (such as ORR, PFS, OS, etc.) to toripalimab than patients without these two biomarkers or patients with either TMB high (≥10) mutations/Mb) or lymph node only metastasis alone. TMB high (≥10 mutations/Mb) in combination with lymph node only metastasis could be used to identify mUC patients who are most likely to benefit from an inhibitor selected from anti-PD-1 antibody (such as toripalimab).

In one embodiment, the candidate is further identified as exhibiting positive PD-L1 expression in a tumor sample. In another embodiment, the candidate is further identified as exhibiting positive PD-L1 expression in immune cells. Patients with TMB high (≥10 mutations/Mb) and PD-L1+ in tumor cells showed significantly better response (such as ORR, PFS, OS, etc.) to toripalimab than patients without these two biomarkers or patients with either TMB high (≥10 mutations/Mb) or PD-L1+ in tumor cells alone. TMB high (≥10 mutations/Mb) in combination with PD-L1+ in tumor cells could be used to identify mUC patients who are most likely to benefit from an inhibitor selected from anti-PD-1 antibody (such as toripalimab).

In one embodiment, the biomarker, for predicting the response of a patient suffering from urothelial carcinoma to the treatment comprising a therapeutically effective amount of an inhibitor selected from anti-PD-1 antibody (such as toripalimab), comprises one or more of the following: tumor mutational burden ≥10 mutations/Mbp; genomic mutations in SMARCA4; genomic mutations in RB1; lymph node only metastasis; genomic mutations in FGFR2 and/or FGFR3 (preferably, FGFR3 gene mutation or FGFR2/FGFR3 gene fusion): genomic mutations in NECTIN4 (preferably, NECTIN4 gene amplification); and positive PD-L1 expression in a tumor sample. Patients having one or more of the above biomarkers exhibit significantly better response to an inhibitor selected from anti-PD-1 antibody (such as toripalimab) than patients without the corresponding biomarkers.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

Patients having mutations in chromatin remodeler SMARCA4 and/or tumor suppressor RB1 exhibit significantly better response (such as ORR, PFS, OS, etc.) to toripalimab than patients with wild type genes.

In a second aspect, the present invention provides a method of treating a patient suffering from urothelial carcinoma, comprising identifying a candidate having mutations in one or more of the following genes occurred in tumor cells: SMARCA4 and RB1; and administering to the candidate a therapeutically effective amount of an inhibitor selected from anti-PD-1 antibody.

SMARCA4 gene encodes a protein which is a part of the large ATP-dependent chromatin-remodeling complex SWI/SNF, and has been identified as a tumor suppressor gene. RB1 gene is a tumor suppressor gene and encodes a negative regulator of the cell cycle; and the protein is encoded by the RB1 gene located on chromosome 13—more specifically, 13q14.1-q14.2.

In one embodiment, the urothelial carcinoma is locally advanced or metastatic urothelial carcinoma. In another embodiment, the urothelial carcinoma is non-metastatic urothelial carcinoma. In one embodiment, the urothelial carcinoma is lower tract urothelial carcinoma (LTUC) originated from bladder or urethral canal. In another embodiment, the urothelial carcinoma is upper tract urothelial carcinoma (UTUC) from renal pelvis or ureter. In one embodiment, the tumor metastasis is lymph node only. In another embodiment, the tumor metastasis is visceral.

In one embodiment, the patient has received a treatment of chemotherapy. In one embodiment, the patient has received a first-line treatment of chemotherapy. In another embodiment, the patient has not received a first-line treatment of chemotherapy before. In another embodiment, the patient has received two lines of chemotherapy. In one embodiment, the patient has received a platinum-based chemotherapy. In another embodiment, the patient has received a non-platinum chemotherapy. In one embodiment, the patient has failed in the previous standard chemotherapy.

In one embodiment, the mutations are somatic mutations. In another embodiment, the mutations are somatic mutations within the coding regions. In another embodiment, the genomic mutations are somatic mutations within the coding regions and non-coding regions.

In one embodiment, the mutations are determined by performing whole exome sequencing. In another embodiment, the mutations are determined by performing whole genome sequencing. In another embodiment, the mutation includes microsatellite stability status, single base substitution, short and long insertions/deletions, copy number variants, and gene rearrangement and fusions, missense mutations, frameshift mutations, nonsense mutation, duplications and repeat expansions. In another embodiment, the mutations are selected from the group consisting of microsatellite stability status, single base substitution, short and long insertions/deletions, copy number variants, and gene rearrangement and fusions.

In one embodiment, the inhibitor is an anti-PD-1 antibody. In another embodiment, the inhibitor is pembrolizumab, nivolumab, tislelizumab, sintilimab, camrelizumab, or cemiplimab. In another embodiment, the inhibitor is toripalimab.

As for other preferred embodiments for the second aspect, please refer to the description in the first aspect.

In a third aspect, the present invention provides a method of predicting the response of a patient suffering from urothelial carcinoma to the treatment comprising a therapeutically effective amount of an inhibitor selected from anti-PD-1 antibody, comprising determining a tumor mutational burden of the patient: comparing the tumor mutational burden with a predetermined reference value, wherein the predetermined reference value is ≥10 mutations/Mbp: concluding that the patient is more likely to respond to the treatment if tumor mutational burden is equal with or higher than the predetermined reference value, compared with the corresponding control group.

In a fourth aspect, the present invention provides a method of predicting the response of a patient suffering from urothelial carcinoma to the treatment comprising a therapeutically effective amount of an inhibitor selected from anti-PD-1 antibody, comprising identifying a patient having mutations in one or more of the following genes occurred in tumor cells: SMARCA4 and RB1; and concluding that the patient is more likely to respond to the treatment, compared with the corresponding control group.

As to the further or preferred embodiments for the method of predicting the response of a patient suffering from urothelial carcinoma to the treatment comprising a therapeutically effective amount of an inhibitor selected from anti-PD-1 antibody (the third aspect and the fourth aspect), please refer to the further or preferred embodiments of the first aspect and the second aspects (method of treating) for details.

### (II) Use of a Composition in the Manufacture of a Medicament for Treating and Use of a Reagent in the Manufacture of a Medicament for Predicting

In one aspect, the present invention provides use of a composition comprising an inhibitor selected from anti-PD-1 antibody in the manufacture of a medicament for treating a patient suffering from urothelial carcinoma, wherein the patient exhibits a high tumor mutational burden of ≥10 mutations/Mbp.

In another aspect, the present invention provides use of a composition comprising an inhibitor selected from anti-PD-1 antibody in the manufacture of a medicament for treating a patient suffering from urothelial carcinoma, wherein the patient has mutations in one or more of the following genes occurred in tumor cells: SMARCA4 and RB1.

In another aspect, the present invention provides use of a reagent for determining a tumor mutational burden in the manufacture of a medicament for predicting the response of the patient suffering from urothelial carcinoma to the treatment comprising a therapeutically effective amount of an inhibitor selected from anti-PD-1 antibody, wherein the predicting comprises determining a tumor mutational burden of the patient; comparing the tumor mutational burden with a predetermined reference value, wherein the predetermined reference value is ≥10 mutations/Mbp: concluding that the patient is more likely to respond to the treatment if tumor mutational burden is equal with or higher than the predetermined reference value, compared with the corresponding control group.

In another aspect, the present invention provides use of a reagent for determining mutations in the manufacture of a medicament for predicting the response of the patient suffering from urothelial carcinoma to the treatment comprising a therapeutically effective amount of an inhibitor selected from anti-PD-1 antibody, wherein the predicting comprises identifying a patient having mutations in one or more of the following genes occurred in tumor cells: SMARCA4 and RB1; and concluding that the patient is more likely to respond to the treatment, compared with the corresponding control group.

Refer to section (I) for the further or preferred embodiments of the above aspects.

### (III) Composition for Use in the Treatment and Reagent for Use in Predicting

In one aspect, the present invention provides a composition comprising an inhibitor selected from anti-PD-1 antibody for use in the treatment of a patient suffering from urothelial carcinoma, wherein the patient exhibits a high tumor mutational burden of ≥10 mutations/Mbp.

In another aspect, the present invention provides a composition comprising an inhibitor selected from anti-PD-1 antibody for use in the treatment of a patient suffering from urothelial carcinoma, wherein the patient has mutations in one or more of the following genes occurred in tumor cells: SMARCA4 and RB1.

In another aspect, the present invention provides a reagent for determining a tumor mutational burden for use in predicting the response of the patient suffering from urothelial carcinoma to the treatment comprising a therapeutically effective amount of an inhibitor selected from anti-PD-1 antibody, wherein the predicting comprises determining a tumor mutational burden of the patient: comparing the tumor mutational burden with a predetermined reference value, wherein the predetermined reference value is ≥10 mutations/Mbp: concluding that the patient is more likely to respond to the treatment if tumor mutational burden is equal with or higher than the predetermined reference value, compared with the corresponding control group.

In another aspect, the present invention provides a reagent for determining mutations for use in predicting the response of the patient suffering from urothelial carcinoma to the treatment comprising a therapeutically effective amount of an inhibitor selected from anti-PD-1 antibody, wherein the predicting comprises identifying a patient having mutations in one or more of the following genes occurred in tumor cells: SMARCA4 and RB1; and concluding that the patient is more likely to respond to the treatment, compared with the corresponding control group. In one embodiment, the urothelial carcinoma is locally advanced or metastatic urothelial carcinoma. In another embodiment, the inhibitor is toripalimab.

Refer to section (I) for the further or preferred embodiments of the above aspects.

### (IV) Composition Comprising an Inhibitor as the Effective Ingredient for Treating and Composition Comprising a Reagent as the Effective Ingredient for Predicting

In one aspect, the present invention provides a composition comprising an inhibitor selected from anti-PD-1 antibody as the effective ingredient for treating a patient suffering from urothelial carcinoma, wherein the patient exhibits a high tumor mutational burden of ≥10 mutations/Mbp.

In another aspect, the present invention provides a composition comprising an inhibitor selected from anti-PD-1 antibody as the effective ingredient for treating a patient suffering from urothelial carcinoma, wherein the patient has mutations in one or more of the following genes occurred in tumor cells: SMARCA4 and RB1. In one embodiment, the urothelial carcinoma is locally advanced or metastatic urothelial carcinoma. In another embodiment, the inhibitor is toripalimab.

In another aspect, the present invention provides a composition comprising a reagent for determining a tumor mutational burden as the effective ingredient for predicting the response of the patient suffering from urothelial carcinoma to the treatment comprising a therapeutically effective amount of an inhibitor selected from anti-PD-1 antibody, wherein the predicting comprises determining a tumor mutational burden of the patient: comparing the tumor mutational burden with a predetermined reference value, wherein the predetermined reference value is ≥10 mutations/Mbp; concluding that the patient is more likely to respond to the treatment if tumor mutational burden is equal with or higher than the predetermined reference value, compared with the corresponding control group.

In another aspect, the present invention provides a composition comprising a reagent for determining mutations as the effective ingredient for predicting the response of the patient suffering from urothelial carcinoma to the treatment comprising a therapeutically effective amount of an inhibitor selected from anti-PD-1 antibody, wherein the predicting comprises identifying a patient having mutations in one or more of the following genes occurred in tumor cells: SMARCA4 and RB1; and concluding that the patient is more likely to respond to the treatment, compared with the corresponding control group. In one embodiment, the urothelial carcinoma is locally advanced or metastatic urothelial carcinoma. In another embodiment, the inhibitor is toripalimab.

Refer to section (I) for the further or preferred embodiments of the above aspects.

### (V) DEFINITIONS AND ABBREVIATIONS

As used throughout the specification and appended claims, the following abbreviations apply:
ORR objective response rates
PFS progression-free survival
OS overall survival
DCR disease control rate
DOR duration of response
PK pharmacokinetics
TMB tumor mutational burden
WES whole exome sequencing
ICI immune checkpoint inhibitors
IHC immunohistochemistry
TPS tumor proportion score
IC immune cell
TC tumor cells
PBMC peripheral blood mononuclear cells
CI confidence interval
AE adverse event
PD-1 programmed death 1
PD-L1 programmed cell death 1 ligand 1

Certain technical and scientific terms are specifically defined below, so that the invention may be more readily understood. Unless specifically defined elsewhere in this document, all other technical and scientific terms used herein have the meaning commonly understood by one of ordinary skill in the art to which this invention belongs.

As used herein, “or” indicates either or both possibilities, unless the context clearly dictates one of the indicated possibilities.

As used herein, including the appended claims, the singular forms of words such as “a”, “an” and “the” include their corresponding plural references unless the context clearly dictates otherwise.

As used herein, the term “tumor mutational burden” refers to the number or rate of mutations in a tumor sample.

As used herein, the term “genomic mutation” refers to permanent change in the DNA sequence. In some embodiments, mutations range in size from a single DNA building block (DNA base) to a large segment of a chromosome. In some embodiments, mutations can include microsatellite stability status, missense mutations, frameshift mutations, nonsense mutation, insertions, deletions, duplications and repeat expansions, copy number variants, and gene rearrangement and fusions. In some embodiments, a missense mutation is a change in one DNA base pair that results in the substitution of one amino acid for another in the protein made by a gene. In some embodiments, a nonsense mutation is also a change in one DNA base pair. Instead of substituting one amino acid for another, however, the altered DNA sequence prematurely signals the cell to stop building a protein. In some embodiments, an insertion changes the number of DNA bases in a gene by adding a piece of DNA. In some embodiments, a deletion changes the number of DNA bases by removing a piece of DNA. In some embodiments, small deletions may remove one or a few base pairs within a gene, while larger deletions can remove an entire gene or several neighboring genes. In some embodiments, a duplication consists of a piece of DNA that is abnormally copied one or more times. In some embodiments, frameshift mutations occur when the addition or loss of DNA bases changes a gene's reading frame. A reading frame consists of groups of 3 bases that each code for one amino acid. In some embodiments, a frameshift mutation shifts the grouping of these bases and changes the code for amino acids. In some embodiments, insertions, deletions, and duplications can all be frameshift mutations. In some embodiments, a repeat expansion is another type of mutation. In some embodiments, nucleotide repeats are short DNA sequences that are repeated a number of times in a row.

As used herein, the term “objective response” refers to size reduction of a cancerous mass by a defined amount. In some embodiments, the cancerous mass is a tumor.

As used herein, the term “objective response rate” (ORR) has its art-understood meaning referring to the proportion of patients with tumor size reduction of a predefined amount and for a minimum time period. In some embodiments, duration of response is usually measured from the time of initial response until documented tumor progression. In some embodiments, ORR involves the sum of partial responses plus complete responses.

As used herein, the term “progression free survival” (PFS) has its art-understood meaning relating to the length of time during and after the treatment of a disease, such as cancer, that a patient lives with the disease but it does not get worse. In some embodiments, measuring the progression-free survival is utilized as an assessment of how well a new treatment works. In some embodiments, PFS is determined in a randomized clinical trial. In some such embodiments, PFS refers to time from randomization until objective tumor progression and/or death.

As used herein, the term “response/respond” may refer to an alteration in a subject's condition that occurs as a result of or correlates with treatment. In some embodiments, a response is or comprises a beneficial response. In some embodiments, a beneficial response may include stabilization of the condition (e.g., prevention or delay of deterioration expected or typically observed to occur absent the treatment), amelioration (e.g., reduction in frequency and/or intensity) of one or more symptoms of the condition, and/or improvement in the prospects for cure of the condition, etc. In some embodiments, a response is or comprises a clinical response. In some embodiments, presence, extent, and/or nature of response may be measured and/or characterized according to particular criteria: in some embodiments, such criteria may include clinical criteria and/or objective criteria.

As used herein, the term “wild-type” has its art-understood meaning that refers to an entity having a structure and/or activity as found in nature in a “normal” (as contrasted with mutant, diseased, altered, etc.) state or context. Those of ordinary skill in the art would appreciate that wild-type genes and polypeptides often exist in multiple different forms (e.g., alleles).

As used herein, the term “somatic mutations” comprises DNA alterations in non-germline cells and commonly occur in cancer cells.

As used herein, the terms “antibody”, or “antigen-binding fragment thereof”, which may be used interchangeably, refer to polypeptide(s) capable of binding to an epitope. In some embodiments, an antibody is a full-length antibody, and in some embodiments, is less than full length but includes at least one binding site (comprising at least one, and preferably at least two sequences with structure of antibody “variable regions”). In some embodiments, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, humanized, fully human antibodies, chimeric antibodies, scFv, etc.

As used therein, the term “anti-PD-1 antibody” refers to any chemical compound or biological molecule which can bind to PD-1 receptor, and can block binding between PD-L1 expressed on tumor cells and PD-1 expressed on immune cells (such as T. B, or NK cells), and preferred can also block binding between PD-L2 expressed on tumor cells and PD-1 expressed on immune cells.

As used herein, unless defined otherwise, when refers to “anti-PD-1 antibody”, the term includes the antigen-binding fragment thereof.

The anti-PD-1 antibody wherein is suitable to any use, method, reagent, or composition of the present invention, can block the binding between PD-1/2 and PD-1, and can inhibit PD-1 signal transduction, to result in immunosuppressive effect. Any use, method, reagent, or composition disclosed herein, wherein anti-PD-1 antibody includes full-length antibody and any antigen-binding moieties or fragments which can bind PD-1 and have similar function properties as a full-length Ab in inhibiting binding with receptor and upregulating immune system.

In some embodiment, the anti-PD-1 antibody or antigen-binding fragment thereof is an anti-PD-1 antibody or antigen-binding fragment that competitive cross-binding human PD-1 with toripalimab. Preferred, in some embodiment, wherein in the use, method, reagent, or composition of the present invention, the PD-1 antibody is a monoclonal antibody or antigen-binding fragment thereof, it comprises at least one of CDR sequences set forth in SEQ ID NOs: 1, 2, 3, 4, 5, or 6. More preferred, in some embodiment, wherein in the use, method, reagent, or composition of the present invention, the PD-1 antibody is a monoclonal antibody or antigen-binding fragment thereof, it comprises LCDR sequences set forth in SEQ ID NOs: 1, 2, and 3, and HCDR sequences set forth in SEQ ID NOs: 4, 5, and 6. Preferred further, in some embodiment, wherein in the use, method, reagent, or composition of the present invention, the PD-1 antibody is a monoclonal antibody or antigen-binding fragment thereof, it comprises a light chain sequence set forth in SEQ ID NO: 9, and/or a heavy chain sequence set forth in SEQ ID NO:10 (toripalimab).

Thus, for example, in some embodiments, the exemplary anti-PD-1 antibody or antigen-binding fragment that binds to PD-1 provided herein, the amino acid sequences of the LCDR1, LCDR2 and LCDR3 of the light chain CDR and the amino acid sequences of the HCDR1, HCDR2 and HCDR3 of the heavy chain CDR are listed as following:

| | |
|---|---|
| LCDR1 | SEQ ID NO: 1 |
| LCDR2 | SEQ ID NO: 2 |
| LCDR3 | SEQ ID NO: 3 |
| HCDR1 | SEQ ID NO: 4 |
| HCDR2 | SEQ ID NO: 5 |
| HCDR3 | SEQ ID NO: 6 |

The anti-PD-1 antibody that binds to PD-1 and can be used in any use, method, reagent, or composition of the present invention are elaborated in international application WO2014206107.

In some embodiments, the anti-PD-1 antibody which can be used in any use, method, reagent, or composition of the present invention further comprises nivolumab, pembrolizumab, toripalimab, sintilimab, camrelizumab, tislelizumab and cemiplimab, or a combination thereof.

As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal nasal, oral, rectal, subcutaneous, sublingual, topical, tracheal (including by intratracheal instillation), transdermal, vaginal and vitreal.

Reference herein to “one embodiment” or “another embodiment” means that a particular feature, structure, or characteristic described in conjunction with the embodiment is included in at least one embodiment. Therefore, the phrases “in one embodiment” or “in another embodiment” appearing in various places herein do not necessarily all refer to the same embodiment. In addition, specific features, structures, or characteristics may be combined in one or more embodiments in any suitable manner.

### Examples

### 1. Methods and Materials

### 1.1 Patients and Study Design

This study is a phase II, multi-center, single arm, open-label, clinical trial (NCT03113266) evaluating the safety and clinical activity of toripalimab in patients with locally advanced or metastatic urothelial carcinoma after failure of standard therapy. The study protocol and all amendments were approved by the institutional ethics committees of all participating centers. This study was conducted in accordance with the Declaration of Helsinki and the international standards of good clinical practice.

Eligible patients were at least 18 years old with pathologically confirmed locally advanced or metastatic urothelial carcinoma who were previously treated with systemic therapy. Patients must have at least one measurable lesion per Response Evaluation Criteria in Solid Tumors (RECIST) version 1.1 at baseline, with Eastern Cooperative Oncology Group (ECOG) performance status of 0 or 1, adequate organ and bone marrow function, and willingness to provide consent for biopsy samples. Exclusion criteria included history of autoimmune diseases, ongoing infections, or prior anti-PD-1/PD-L1/PD-L2 based immunotherapies.

### 1.2 Treatment and End Points

Patients received toripalimab 3 mg/kg once every two weeks via intravenous infusion until disease progression, intolerable toxicity, or voluntary withdrawal of informed consent. Adverse events were monitored continuously and graded according to the National Cancer Institute Common Terminology Criteria (CTCAE) version 4.0. In this study, “Definitely related”, “Probably related”, and “Possibly related” were classified as “treatment related” AE (TRAE). “Possibly unrelated” and “Definitely unrelated” were classified as “treatment unrelated”. Radiographic imaging was performed before treatment, then once every 8 weeks in the first year and once every 12 weeks from the second year until disease progression and evaluated by investigators using both RECIST v1.1. Patients who initially developed progressive disease per RECIST v1.1 were allowed to continue therapy if the investigator considered patients benefiting from further treatment.

The primary endpoint of this study was safety and clinical efficacy by objective response rate (ORR) determined by independent radiologic review committee per RECIST v1.1. The secondary endpoints included pharmacokinetics (PK) and immunogenicity of toripalimab (anti-drug antibody, ADA), disease control rate (DCR), duration of response (DOR), progression free survival (PFS), and overall survival (OS).

### 1.3 PD-L1 Expression Analysis in Tumor Biopsies

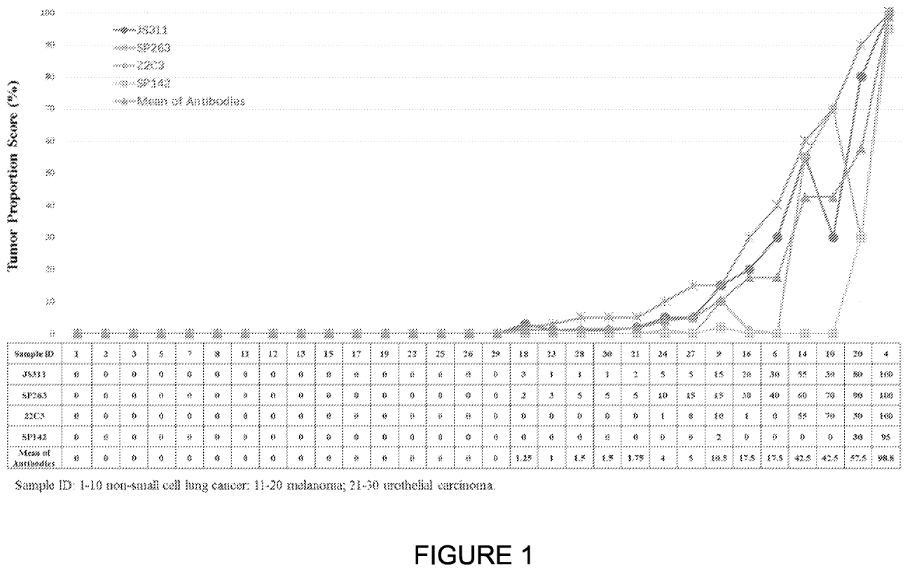

Archival or fresh tumor biopsy samples were obtained from patients prior to treatment. PD-L1 expression was evaluated by immunohistochemistry (IHC) staining with JS311 antibody using a validated staining assay on Ventana Benchmark Ultra platform in a central lab. JS311 is monoclonal rabbit anti-human PD-L1 antibody developed for IHC staining 1. Cross correlation study had been performed between different PD-L1 IHC Assays and JS311 showed similar PD-L1 staining patterns and scores with SP263 antibody (rabbit monoclonal primary antibody. Roche) in tumor biopsies from various cancer types including urothelial carcinoma (FIG. 1). PD-L1 positive was defined as tumor proportion score (TPS) ≥1%, namely the presence of membrane staining of any intensity in ≥1% of tumor cells (TC). PD-L1 expression on immune cell (IC) was also evaluated. PD-L1 IC+ was defined as immune cell positive staining ≥1%.

### 1.4 Tumor Mutational Burden Analysis

Whole exome sequencing (WES) was performed with SureSelect Human All Exon V6 kit (Agilent) on tumor biopsies and matched peripheral blood mononuclear cells (PBMC) samples. Genomic alterations including microsatellite stability status, single base substitution (SNV), short and long insertions/deletions (INDELs), copy number variants (CNV), and gene rearrangement and fusions were assessed. The tumor mutational burden (TMB) was determined by analyzing somatic mutations (including the above mentioned genomic alterations) per mega-base (Mb).

### 1.5 Statistical Analysis

At a one-sided significance level of 0.025, a total of 150 patients could provide 91% power to demonstrate the efficacy of toripalimab at targeted ORR of 20% versus 10% for alternative 2^{nd} line therapy using Clopper-Pearson method. A 150-patients sample size was thus planned for this study and 151 patients were enrolled.

Safety analysis included all patients who received at least 1 dose of the study drug (n=151). ORR and its 95% exact confidence interval (CI) were determined by Clopper and Pearson methodology. Fisher's exact test was used to compute two-tailed P values from contingency tables. PFS and OS were plotted using the Kaplan-Meier method, with median and corresponding two-sided 95% CI. Statistics analyses were performed with SAS version 9.4 or GraphPad Prism software.

### 2. Results

### 2.1 Patient Population

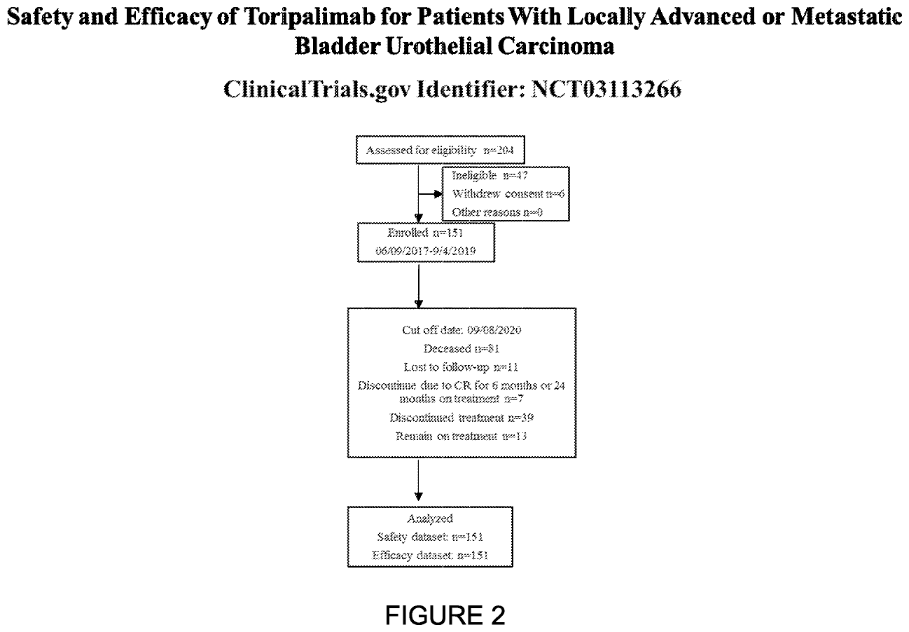

Between May 2017 and September 2019, 151 patients were enrolled from 15 participating centers (FIG. 2). Baseline demographic and clinical characteristics are summarized in Table 1. One hundred and thirty-two (87%) patients had visceral metastasis at enrollment, including 50% with pulmonary metastasis, 29% with bone metastasis, and 15% with hepatic metastasis. The primary tumor sites included 47% upper urinary tract and 52% lower urinary tract. Forty-eight (32%) patients had positive PD-L1 expression on tumor biopsies. All patients had received prior systemic chemotherapy, including 95% platinum-based therapy and 5% non-platinum chemotherapy.

### 2.2 Treatment Related Toxicity

By the cutoff date of Sep. 15, 2020, 12 months after the last enrollment, patients received a median of 8 doses of toripalimab (range 1 to 66 doses). The median follow up was 10.5 months. We did not identify new safety concerns with toripalimab monotherapy compared with ICIs in the same class. One hundred and twenty-eight (84.8%) patients experienced treatment related adverse events (TRAEs). Common TRAEs (>10%) were listed in Table 2. Grade 3 and above TRAEs occurred in 30 (19.9%) patients, including 27 (17.9%) patients with grade 3 and 3 (2.0%) with grade 4 TRAE (Table 3). There was no Grade 5 TRAE. Permanent discontinuation of toripalimab due to TRAEs occurred in 5 (3.3%) patients, and dose interruption due to TRAEs occurred in 22 (14.6%) patients. Two patients developed infusion reactions (one Grade 1 and one Grade 2), both of which were relieved by symptomatic treatment. Immune-related adverse events (irAEs) included 15 (9.9%) hypothyroidism, 12 (7.9%) hyperthyroidism, 4 (2.6%) abnormal liver function, 2 (1.3%) interstitial lung disease, 2 (1.3%) adrenal insufficiency, 1 (0.7%) autoimmune hepatitis, and 1 (0.7%) myocarditis.

TABLE 1
Summary of baseline demographic and clinical characteristics.

| Characteristics | Value | N (%) | |
|---|---|---|---|
| Age | N | 151 | |
| | Mean | 60.9 | |
| | Min, Max | 28.0, 82.0 | |
| Gender | Male | 101 | (66.9) |
| | Female | 50 | (33.1) |
| ECOG | 0 | 66 | (43.7) |
| | 1 | 85 | (26.3) |
| TNM stage | IIIb | 1 | (0.7) |
| | IV | 150 | (99.3) |
| Tumor metastasis | Lymph node only | 19 | (12.6) |
| | Visceral | 132 | (87.4) |
| Primary tumor sites ^{a} | Upper urinary track | 71 | (47.0) |
| | Lower urinary track | 78 | (51.7) |
| | Others | 2 | (1.3) |
| Prior lines of treatment ^{b} | Adjuvant | 14 | (9.3) |
| | 1 L | 105 | (69.5) |
| | 2 L+ | 32 | (21.2) |
| Prior chemotherapy | Platinum based | 143 | (94.7) |
| | Others | 8 | (5.3) |
| PD-L1 status ^{c} | Positive | 48 | (31.8) |
| | Negative | 96 | (63.6) |
| | Unknown | 7 | (4.6) |

ECOG, Eastern Cooperative Oncology Group; TNM, Tumor, node, metastasis staging system;
^{a} Upper urinary track includes renal pelvis and ureter; Lower urinary track includes bladder and urethral canal.
^{b} Adjuvant setting included 14 patients who experienced progressive disease within 6 months of the last adjuvant or neoadjuvant chemotherapy.
^{c} Positive defined as ≥1% of tumor cells expressing PD-L1 by JS311 IHC staining.

TABLE 2
Common (>10%) treatment-related adverse events (TRAEs) in the study (N = 151).

| N (%) | | Grade 1 | | Grade 2 | | Grade 3 | Grade 4 | Grade 5 |
|---|---|---|---|---|---|---|---|---|
| All TRAES | 128 (84.8) | 31 | (20.5) | 67 | (44.4) | 27 (17.9) | 3 (2.0) | 0 |
| Blood triglycerides | 36 (23.8) | 23 | (15.2) | 12 | (7.9) | 1 (0.7) | 0 | 0 |
| increased | | | | | | | | |
| Proteinuria | 29 (19.2) | 9 | (6.0) | 20 | (13.2) | 0 | 0 | 0 |
| Anemia | 27 (17.9) | 11 | (7.3) | 11 | (7.3) | 4 (2.6) | 0 | 0 |
| Asthema | 26 (17.2) | 21 | (13.9) | 4 | (2.6) | 1 (0.7) | 0 | 0 |
| Leukopenia | 25 (16.6) | 18 | (11.9) | 6 | (4.0) | 1 (0.7) | 0 | 0 |
| Blood cholesterol | 21 (13.9) | 19 | (12.6) | 1 | (0.7) | 1 (0.7) | 0 | 0 |
| increased | | | | | | | | |
| Blood creatine | 21 (13.9) | 12 | (7.9) | 7 | (4.6) | 1 (0.7) | 1 (0.7) | |
| phosphokinase increased | | | | | | | | |
| Blood glucose increased | 20 (13.2) | 14 | (9.3) | 3 | (2.0) | 3 (2.0) | 0 | 0 |
| Apolipoprotein B | 17 (11.3) | 17 | (11.3) | 0 | | 0 | 0 | 0 |
| increased | | | | | | | | |
| AST increased | 17 (11.3) | 14 | (9.3) | 1 | (0.7) | 2 (1.3) | 0 | 0 |
| Rash | 17 (11.3) | 11 | (7.3) | 5 | (3.3) | 1 0.7) | 0 | 0 |
| ALT increased | 16 (10.6) | 13 | (8.6) | 2 | (1.3) | 1 (0.7) | 0 | 0 |

“Definitely related”, “Probably related”, and “Possibly related” were classified as “treatment related” AE (TRAE). “Possibly unrelated” and “Definitely unrelated” were classified as “treatment unrelated”. ALT, alanine aminotransferase; AST, aspartate transaminase.

TABLE 3
Grade 3 and above treatment-related adverse events in the study.

| AE Terms | Grade 2 | Grade 3 | Grade 4 | Grade 5 |
|---|---|---|---|---|
| All grade 3 and above | 30 (19.9) | 27 (17.9) | 3 (2.0) | 0 |
| Anemia | 4 (2.6) | 4 (2.6) | 0 | 0 |
| Blood glucose increased | 3 (2.0) | 3 (2.0) | 0 | 0 |
| AST increased | 2 (1.3) | 2 (1.3) | 0 | 0 |
| Neutropenia | 2 (1.3) | 2 (1.3) | 0 | 0 |
| Blood sodium decreased | 2 (1.3) | 2 (1.3) | 0 | 0 |
| Blood creatine phosphokinase | 2 (1.3) | 1 (0.7) | 1 (0.7) | 0 |
| increased | | | | |
| Hypertension | 2 (1.3) | 2 (1.3) | 0 | 0 |
| Amylase increased | 2 (1.3) | 1 (0.7) | 1 (0.7) | 0 |
| Lymphopenia | 2 (1.3) | 2 (1.3) | 0 | 0 |
| Rash | 1 (0.7) | 1 (0.7) | 0 | 0 |
| ALT increased | 1 (0.7) | 1 (0.7) | 0 | 0 |
| Diarrhoea | 1 (0.7) | 1 (0.7) | 0 | 0 |
| Thrombocytopenia | 1 (0.7) | 1 (0.7) | 0 | 0 |
| Blood potassium decreased | 1 (0.7) | 1 (0.7) | 0 | 0 |
| Hepatic function abnormal | 1 (0.7) | 1 (0.7) | 0 | 0 |
| GGT increased | 1 (0.7) | 1 (0.7) | 0 | 0 |
| Urinary tract infection | 1 (0.7) | 1 (0.7) | 0 | 0 |
| Adrenal insufficiency | 1 (0.7) | 1 (0.7) | 0 | 0 |
| Hypertension | 1 (0.7) | 1 (0.7) | 0 | 0 |
| Lipase increased | 1 (0.7) | 0 | 1 (0.7) | 0 |
| White blood cell count | 1 (0.7) | 1 (0.7) | 0 | 0 |
| increased | | | | |
| Autoimmune hepatitis | 1 (0.7) | 1 (0.7) | 0 | 0 |
| Cystitis | 1 (0.7) | 1 (0.7) | 0 | 0 |
| Hepatic failure | 1 (0.7) | 1 (0.7) | 0 | 0 |
| Myositis | 1 (0.7) | 1 (0.7) | 0 | 0 |
| Psoriasis | 1 (0.7) | 1 (0.7) | 0 | 0 |
| Type 2 diabetes mellitus | 1 (0.7) | 1 (0.7) | 0 | 0 |
| Blood triglycerides increased | 1 (0.7) | 1 (0.7) | 0 | 0 |
| Asthenia | 1 (0.7) | 1 (0.7) | 0 | 0 |
| Leukopenia | 1 (0.7) | 1 (0.7) | 0 | 0 |
| Blood cholesterol increased | 1 (0.7) | 1 (0.7) | 0 | 0 |

### 2.3 Antitumor Activity

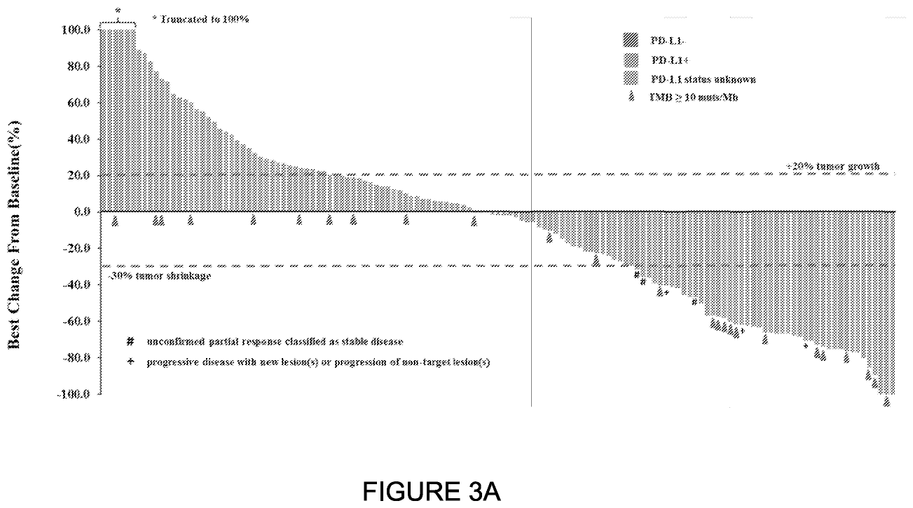

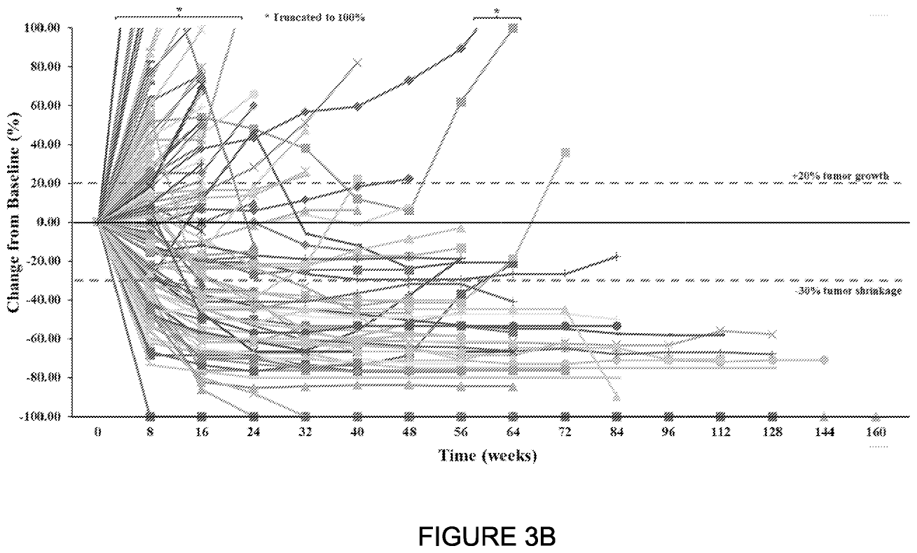

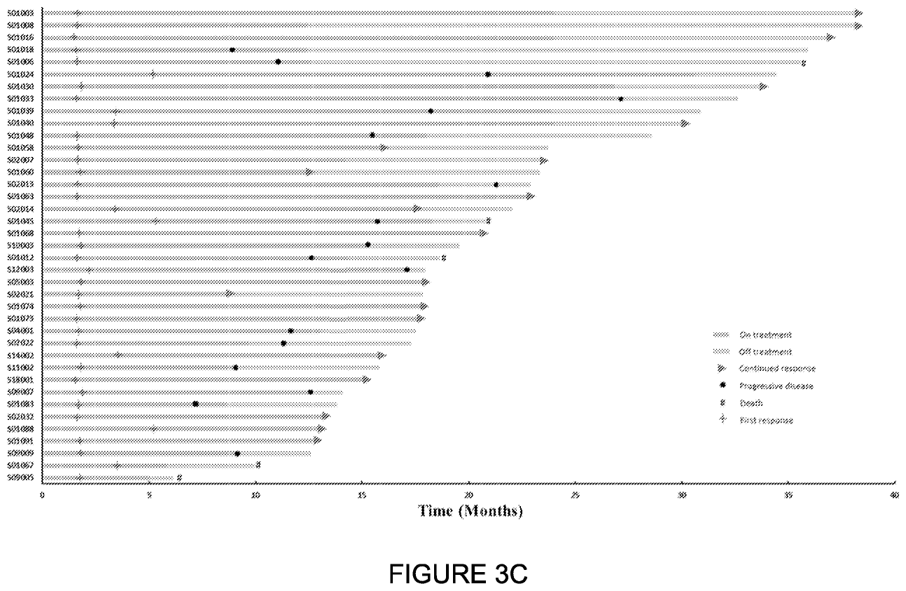

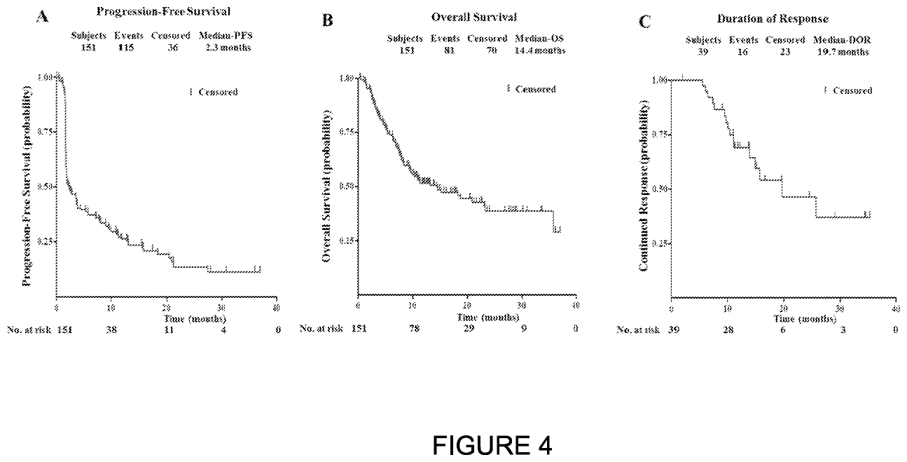

As of Sep. 15, 2020, 81 (54%) patients died, 46 (30%) discontinued treatment, 11 (7%) lost in follow-up and 13 (9%) remained on treatment. The median treatment duration was 3.3 months (range 0.03 to 30.7 months). Among the intent-to-treat (ITT) population (n=151), the confirmed ORR was 25.8% (95% CI: 19.1 to 33.6) and the DCR was 45.0% (95% CI 36.9 to 53.3) as assessed by IRC per RECIST v1.1 (Table 4 and FIG. 3). Patients who failed prior platinum-based chemotherapy (n=143) had a similar ORR of 25.9% to toripalimab monotherapy. The ORRs were similar in patients with primary tumor sites in upper urinary tract (n=71) and in lower urinary tract (n=78), 26.8% versus 24.4%. The responses were durable as the median DOR was 19.7 months (95% CI: 13.9 to NE) (FIG. 4C). The median time to response was 1.8 months (95% CI: 1.7-1.8). For the ITT population, the median PFS was 2.3 months (95% CI: 1.8 to 3.6) and the median OS was 14.4 months (95% CI: 9.3 to 23.1) (FIG. 4).

TABLE 4
Clinical efficacy assessed by independent review
committee (IRC) and investigator in the intent-
to-treat (ITT) population per RECIST v1.1.

| | |
|---|---|
| IRC (n = 151) | Investigator (n = 151) |
| n (%) | n (%) |

| | | | | |
|---|---|---|---|---|
| CR | 2 | (1.3%) | 4 | (2.6%) |
| PR | 37 | (24.5%) | 32 | (21.2%) |
| SD | 29 | (19.2%) | 33 | (21.9%) |
| PD | 68 | (45.0%) | 69 | (45.7%) |
| NE | 15 | (9.9%) | 13 | (8.6%) |
| ORR (%) ^{a} | 39 | (25.8%) | 36 | (23.8%) |

| | | |
|---|---|---|
| 95% CI | 19.1%, 33.6% | 17.3%, 31.4% |

| | | | | |
|---|---|---|---|---|
| DCR (%) ^{b} | 68 | (45.0%) | 69 | (45.7%) |

| | | |
|---|---|---|
| 95% CI | 36.9%, 53.3% | 37.6%, 54.0% |

CR, complete response; PR, partial response; SD, stable disease; PD, progressive disease; NE, not evaluable; ORR, objective response rate; DCR, disease control rate; CI, confidence interval.
^{a} ORR = (CR + PR)/Total*100%.
^{b} DCR = (CR + PR + SD)/Total*100%.

By cutoff date of Sep. 8, 2021, no emergent of new safety signal was identified compared with the previous one-year report. By the cutoff date, 3 CR, 37 PR and 28 SD were observed among the ITT population for an ORR of 26.5% and a DCR of 45.0% as assessed by the IRC. The response was durable as the median duration of response was 25.8 months. The median OS was 14.6 months.

### 2.4 PD-L1 Expression in Tumor

Tumor biopsy samples were obtained from all 151 patients. PD-L1 IHC staining identified 48 (32%) positive, 96 (64%) negative, and 7 (5%) status unknown.

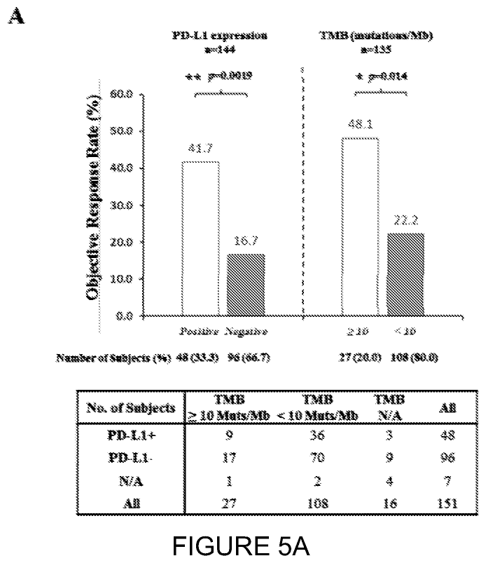

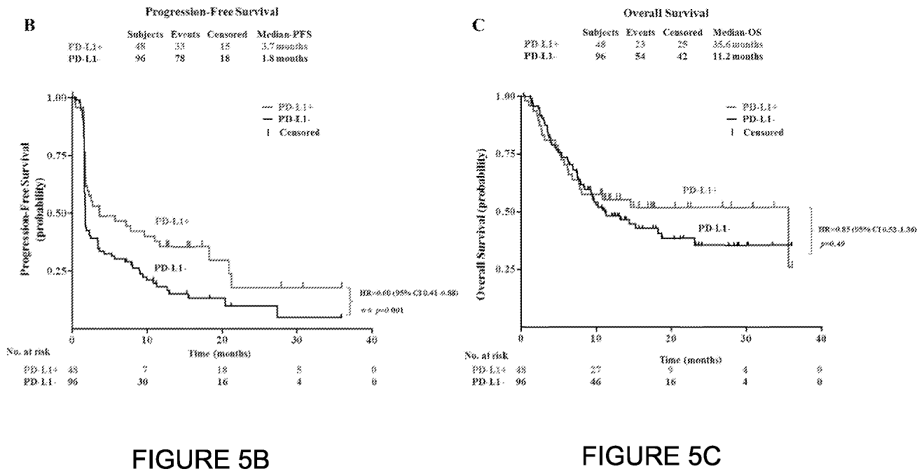

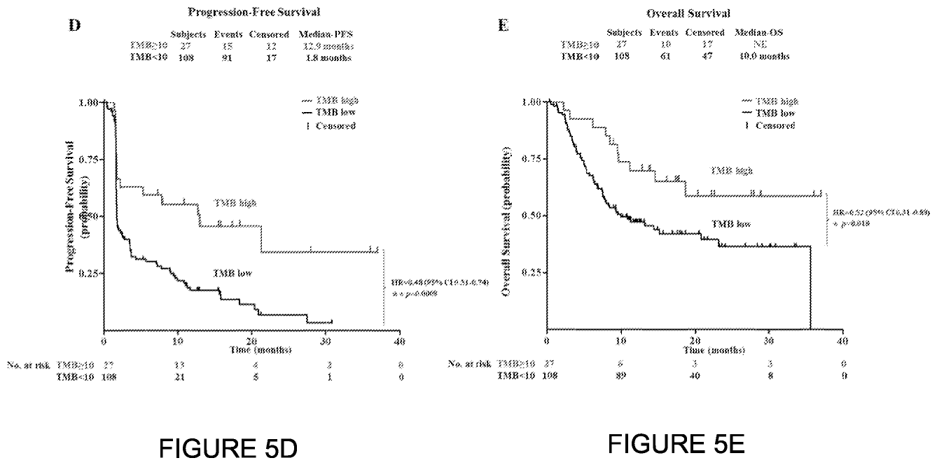

PD-L1+ patients, defined by tumor cell (TC) positive staining ≥1%, had significantly better ORR and PFS than PD-L1− patients, ORR 41.7% versus 16.7%, p-0.0019; median PFS 3.7 versus 1.8 months, HR=0.60 (95% CI 0.41-0.88), p=0.001 (FIG. 5). Furthermore, PD-L1+ patients had numerically better overall survival than PD-L1− patients, median OS 35.6 vs 11.2 months, HR=0.85 (95% CI 0.53 to 1.36), p=0.49. However, the difference was not statistically significant.

PD-L1 expression by immune cell (IC) was also evaluated. PD-L1 IC+ patients defined by IC positive staining ≥1%, accounted for 72% (109/151) of the ITT population. PD-L1 IC+ patients also had significantly better ORR than PD-L1 IC− patients, 30.3% versus 8.6%, p=0.012. The vast majority 96% (46/48) of PD-L1 TC+ samples were also PD-L1 IC+. Patients with PD-L1 IC+ but PD-L1 TC− expression had an ORR of 22.2%, whereas PD-L1 TC− and IC− patients have an ORR of only 6.1% (Table 5).

TABLE 5
PD-L1 JS311 IHC staining results and clinical efficacy. Tumor
biopsies were obtained from all 151 patients and stained
by JS311 antibody for PD-L1 expression. PD-L1 expression
status from 7 (5%) samples could not be determined.

| | | |
|---|---|---|
| PD-L1 expression status | n (%) | ORR % (95% CI) |

| | | | | |
|---|---|---|---|---|
| TC+ | 48 | (32%) | 41.7 | (27.6, 56.8) |
| TC− | 96 | (64%) | 16.7 | (9.8, 25.7) |
| IC+ | 109 | (72%) | 30.3 | (21.8, 39.8) |
| IC− | 35 | (23%) | 8.6 | (1.8, 23.1) |
| TC+ and IC+ | 46 | (30%) | 41.3 | (27.0, 56.8) |
| TC+ and IC− | 2 | (1%) | 50.0 | (1.3, 98.7) |
| TC− and IC+ | 63 | (42%) | 22.2 | (12.7, 34.5) |
| TC− and IC− | 33 | (22%) | 6.1 | (0.7-20.2) |
| TC+ or IC+ | 111 | (74%) | 30.6 | (22.2, 40.1) |
| Any status | 151 | (100%) | 25.8 | (19.1, 33.6) |

ORR, objective response rate; TC, tumor cell; IC, immune cell.
PD-L1 TC+ is defined as tumor cell (TC) positive staining ≥1%.
PD-L1 IC+ is defined as immune cell (IC) positive staining ≥1%.

### 2.5 Genomic Mutational Analysis and Tumor Mutational Burden

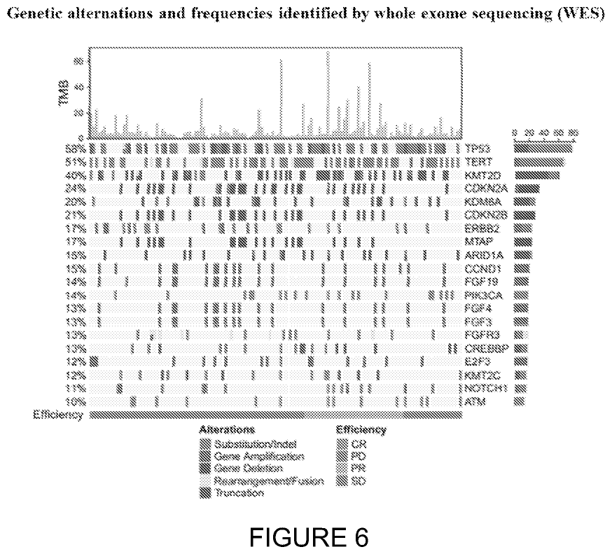

Whole exome sequencing was performed on tumor biopsies and paired PBMCs. Sequencing results were available from 135 patients (FIG. 6). The most frequently altered genes identified in this study included TP53 (58%), TERT (51%), KMT2D (40%), CDKN2A (24%), CDKN2B (21%), KDM2A (20%), ERBB2 (17%), MTAP (17%), ARID1A (15%), CCND1(15%), FGF19 (14%), PIK3CA (14%), FGF4 (13%), FGF3 (13%), FGFR3 (13%), CREBBP (13%), E2F3 (12%), KMT2C (12%), NOTCH1 (11%), ATM1 (10%) and NECTIN4 (9%) (FIG. 6).

Patients having mutations in chromatin remodeler SMARCA4 (n=12) or tumor suppressor RB1 (n=12) exhibited significantly better response to toripalimab than patients with wild type genes. Patients with either mutation had an ORR of 58.3% versus 24.4% for wild type, p=0.019.

The ORR was 30% (6/20) in patients with FGFR3 mutations or FGFR2/FGFR3 gene fusions, and 41.7% (5/12) in patients with NECTIN4 genomic alternations (including 11 NECTIN4 gene amplifications). While 23 patients with ERBB2/HER2 genomic alternations had an ORR of 17.4%, 9 patients with genomic ERBB2/HER2 amplifications had no response to toripalimab.

Tumor mutational burden (TMB) was determined by analyzing somatic mutations within the coding region of the human genome. The median TMB value was 4.1 mutations per million base pairs (Mb) in the cohort. Tumor tissues from 27 (20%) patients harbored more than 10 mutations/Mb. Patients with TMB high (≥10 mutations/Mb) had responded significantly better than patients with TMB low (<10 mutations/Mb) to toripalimab monotherapy, ORR 48.1% versus 22.2%, p=0.014 (FIG. 5A). Importantly, TMB high patients also showed significant survival advantage in both PFS and OS (FIGS. 5D and 5E), median PFS 12.9 versus 1.8 months, HR=0.48 (95% CI 0.31-0.74), p=0.0009 and median OS not reached vs 10.0 months, HR=0.52 (95% CI 0.31 to 0.89), p=0.018. Notably, TMB high population were not enriched in PD-L1+ patients, as 20% of total patients as well as 20% of PD-L1+ patients were also TMB high (FIG. 5A).

Patients with TMB high (≥10 mutations/Mb) and PD-L1+ showed significant high ORR 77.8% (7 out of 9).

### 2.6 Other Biomarkers and Subgroups Analysis

Additional biomarkers or subgroups analyzed for correlation with clinical efficacy included age, gender, baseline ECOG PS score, metastatic status, baseline LDH levels, prior chemotherapy regimen, prior lines of treatments, primary tumor sites and anti-drug antibody (ADA) status (Table 6). Among the subgroups, patients with lymph node only metastasis (n=19) had significantly better ORR than patients with visceral metastasis (n=132), 52.6% versus 22.0%, p=0.0092. The ORRs were 18.3%, 20.9% and 8.7% for patients with pulmonary, bone and hepatic metastasis respectively.

TABLE 6
Biomarker and subgroup analysis for correlation
with clinical efficacy in all 151 patients.

| | | | ORR | 95% CI |
|---|---|---|---|---|
| Characteristics | Value | N | (%) | (%) |
| Age | <65 | 94 | 22.3 | 14.4, 32.1 |
| | ≥65 | 57 | 31.6 | 19.9, 45.2 |
| Gender | Male | 101 | 22.8 | 15.0, 32.2 |
| | Female | 50 | 32.0 | 19.5, 46.7 |
| ECOG | 0 | 66 | 27.3 | 17.0, 39.6 |
| | 1 | 85 | 24.7 | 16.0, 35.3 |
| Primary tumor site ^{a} | Upper urinary track | 71 | 26.8 | 16.9, 38.6 |
| | Lower urinary track | 78 | 24.4 | 15.3, 35.4 |
| | Others | 2 | 50.0 | 1.3, 98.7 |
| Tumor metastasis | Lymph node only | 19 | 52.6 | 28.9, 75.6 |
| | Visceral | 132 | 22.0 | 15.2, 30.0 |
| Prior lines of treatments ^{b} | Adjuvant | 14 | 28.6 | 8.4, 58.1 |
| | 1 L | 105 | 26.7 | 18.5, 36.2 |
| | 2 L+ | 32 | 21.9 | 9.3, 40.0 |
| Prior chemotherapy | Platinum based | 143 | 25.9 | 18.9, 33.9 |
| | Others | 8 | 25.0 | 3.2, 65.1 |
| LDH | >ULN | 28 | 25.0 | 10.7, 44.9 |
| | ≤ULN | 123 | 26.0 | 18.5, 34.7 |
| PD-L1 status ^{c} | Positive | 48 | 41.7 | 27.6, 56.8 |
| | Negative | 96 | 16.7 | 9.8, 25.7 |
| | Unknown | 7 | 42.9 | 9.9, 81.6 |
| ADA status after | Positive | 10 | 30 | 6.7, 65.2 |
| treatment | Negative | 127 | 28.3 | 20.7, 37.0 |
| | N/A | 14 | 0 | NE, NE |
| Total | | 151 | 25.8 | 19.1, 33.6 |

ECOG, Eastern Cooperative Oncology Group; LDH, Lactate dehydrogenase; ULN, upper limit of normal; N/A, not available, NE, not estimable.
^{a} Upper urinary track includes renal pelvis and ureter; Lower urinary track includes bladder and urethral canal.
^{b} Adjuvant setting included 14 patients who experienced progressive disease within 6 months of the last adjuvant or neoadjuvant chemotherapy.
^{c} Positive defined as ≥1% of tumor cells expressing PD-L1 by JS311 IHC staining.

### REFERENCES

1. Wang Z, Ying J, Xu J, et al. Safety, Antitumor Activity, and Pharmacokinetics of Toripalimab, a Programmed Cell Death 1 Inhibitor, in Patients With Advanced Non-Small Cell Lung Cancer: A Phase 1 Trial. JAMA Netw Open 2020; 3(10): e2013770.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-19. (canceled)

20. A method of treating a patient suffering from locally advanced or metastatic urothelial carcinoma, comprising administering a composition comprising an inhibitor selected from anti-PD-1 antibody to the patient, wherein the patient exhibits a high tumor mutational burden of ≥10 mutations/Mbp.

21. The method of claim 20, wherein the patient has received a prior treatment of adjuvant or neoadjuvant chemotherapy.

22. The method of claim 20, wherein the tumor mutational burden is determined by performing whole exome sequencing, or by analyzing genomic mutations selected from the group consisting of microsatellite stability status, single base substitution, short and long insertions/deletions, copy number variants, and gene rearrangement and fusions, wherein the genomic mutations are somatic mutations.

23. The method of claim 22, wherein the patient further has genomic mutations in one or more of the following genes: SMARCA4 and RB1.

24. The method of claim 22, wherein the patient further has FGFR3 gene mutation or FGFR2/FGFR3 gene fusion.

25. The method of claim 22, wherein the patient further has genomic mutations in NECTIN4 gene amplification.

26. The method of claim 20, wherein the patient further exhibits positive PD-L1 expression in a tumor sample, and has lymph node only metastasis.

27. The method of claim 20, wherein the inhibitor is an anti-PD-1 antibody or the antigen binding fragment thereof having the LCDR1, LCDR2, LCDR3 sequences respectively set forth in SEQ ID NOs: 1-3, and the HCDR1, HCDR2, HCDR3 sequences respectively set forth in SEQ ID NOs: 4-6.

28. A method of treating a patient suffering from locally advanced or metastatic urothelial carcinoma, comprising administering a composition comprising an inhibitor selected from anti-PD-1 antibody to the patient, wherein the patient has mutations in one or more of the following genes occurred in tumor cells: SMARCA4 and RB1.

29. The method of claim 28, wherein the patient has received a prior treatment of adjuvant or neoadjuvant chemotherapy.

30. The method of claim 28, wherein the mutations are somatic mutations.

31. The method of claim 28, wherein the inhibitor is an anti-PD-1 antibody or the antigen binding fragment thereof having the LCDR1, LCDR2, LCDR3 sequences respectively set forth in SEQ ID NOs: 1-3, and the HCDR1, HCDR2, HCDR3 sequences respectively set forth in SEQ ID NOs: 4-6.

32. A composition comprising an inhibitor selected from anti-PD-1 antibody as the effective ingredient for treating a patient suffering from locally advanced or metastatic urothelial carcinoma, wherein the patient exhibits a high tumor mutational burden of ≥10 mutations/Mbp and wherein the inhibitor is an anti-PD-1 antibody or the antigen binding fragment thereof having the LCDR1, LCDR2, LCDR3 sequences respectively set forth in SEQ ID NOs: 1-3, and the HCDR1, HCDR2, HCDR3 sequences respectively set forth in SEQ ID NOs: 4-6.

33. The composition of claim 32, wherein the patient has received a prior treatment of chemotherapy.

34. The composition of claim 32, wherein the tumor mutational burden is determined by performing whole exome sequencing or by analyzing genomic mutations selected from the group consisting of microsatellite stability status, single base substitution, short and long insertions/deletions, copy number variants, and gene rearrangement and fusions, wherein the genomic mutations are somatic mutations.

35. The composition of claim 34, wherein the patient further has genomic mutations in one or more of the following genes: SMARCA4 and RB1.

36. The composition of claim 34, wherein the patient further has genomic mutations in FGFR3 gene mutation or FGFR2/FGFR3 gene fusion.

37. The composition of claim 34, wherein the patient further has genomic mutations in NECTIN4 gene amplification.

38. The composition of claim 32, wherein the patient further exhibits positive PD-L1 expression in a tumor sample, and the patient has lymph node only metastasis.

39. A composition comprising an inhibitor selected from anti-PD-1 antibody as the effective ingredient for treating a patient suffering from locally advanced or metastatic urothelial carcinoma, wherein the patient has mutations in one or more of the following genes occurred in tumor cells: SMARCA4 and RB1, and wherein the inhibitor is an anti-PD-1 antibody or the antigen binding fragment thereof having the LCDR1, LCDR2, LCDR3 sequences respectively set forth in SEQ ID NOs: 1-3, and the HCDR1, HCDR2, HCDR3 sequences respectively set forth in SEQ ID NOs: 4-6.

40. The composition of claim 39, wherein the patient has received a prior treatment of chemotherapy.

41. The composition of claim 39, wherein the mutations are somatic mutations.
